(19) United States
(12) Patent Application Publication
(10) Pub. No.: US 2022/0112540 A1
(43) Pub. Date: Apr. 14, 2022

(54) METHODS AND SYSTEMS FOR DISEASE DETECTION

(71) Applicant: AccuraGen Holdings Limited, George Town (KY)

(72) Inventors: Li WENG, Fremont, CA (US); Malek FAHAM, Burlingame, CA (US); Tobias WITTKOP, San Mateo, CA (US); Chia-Hui LIN, San Mateo, CA (US); Ling Fung TANG, San Francisco, CA (US); Johnny WU, Castro Valley, CA (US)

(21) Appl. No.: 17/557,972

(22) Filed: Dec. 21, 2021

Related U.S. Application Data
(63) Continuation of application No. PCT/US2020/039357, filed on Jun. 24, 2020.
(60) Provisional application No. 62/866,432, filed on Jun. 25, 2019.

(51) Int. Cl. C12Q 1/6806; C12Q 1/6886

(52) U.S. Cl. CPC C12Q 1/6806; C12Q 2600/156; C12Q 1/6886

(57) ABSTRACT

Provided herein are methods of determining that a subject has or is at risk of having a disease (e.g., cancer) using nucleic acid molecules derived from a cell-free biological sample of the subject.

### CROSS-REFERENCE

This application is a continuation of PCT International Application Serial No. PCT/US2020/039357 filed Jun. 24, 2020, which claims the benefit of U.S. Provisional Application No. 62/866,432 filed Jun. 25, 2019, which is incorporated herein by reference in its entirety.

### BACKGROUND

Detection of diseases, such as cancer, in individuals via non-invasive methods allows for routine screening of individuals for diseases, such as cancer, resulting in early diagnosis before the disease has worsened or spread, allowing for better treatment outcomes in individuals.

### SUMMARY

Provided herein are methods for determining that a subject has or is at risk of having a disease (e.g., cancer). In one aspect, a method for determining whether a subject has or is at risk of having the disease (e.g., cancer) may comprise obtaining a cell-free biological sample of the subject and using a plurality of nucleic acid molecules derived from the cell-free biological sample to generate a plurality of circular nucleic acid molecules. The plurality of circular nucleic acids or derivatives thereof may then be subjected to sequencing to generate a plurality of sequence reads. The plurality of sequence reads may be processed against a plurality of reference sequence reads to identify at least a subset of the plurality sequence reads as corresponding to at least a subset of the plurality of reference sequence reads, thereby determining that the subject has or is at risk of having the disease. In some cases, generating a plurality of circular nucleic acid molecules comprises ligating ends of each of the plurality of nucleic acid molecules to one another. In some cases, generating a plurality of circular nucleic acid molecules comprises coupling an adapter to a 3′ end, a 5′ end, or both a 5′end and a 3′end of each of the plurality of nucleic acid molecules. In some cases, each of the plurality of circular nucleic acid molecules is subjected to nucleic acid amplification to generate a plurality of amplification products of the plurality of circular nucleic acid molecules, wherein generating a plurality of sequence reads comprises subjecting the plurality of amplification products or derivatives thereof to sequencing to generate a plurality of sequence reads of the plurality of amplification products or derivatives thereof. In some cases, the nucleic acid amplification is effected by a polymerase having strand-displacement activity. In some cases, the nucleic acid amplification is effected by a polymerase that does not have strand-displacement activity. In some cases, the nucleic acid amplification comprises contacting the circular nucleic acid molecule to an amplification reaction mixture comprising random primers. In some cases, the nucleic acid amplification comprises contacting the circular nucleic acid molecule to an amplification reaction mixture comprising one or more primers, each of which specifically hybridizes to a different target sequence via sequence complementarity. In some cases, the plurality of nucleic acid molecules is single stranded. In some cases, the plurality of nucleic acid molecules comprises cell-free DNA or RNA. In some cases, the plurality of nucleic acid molecules is from a tumor. In some cases, the method further comprises processing the sequence to monitor a progression or regression of the disease in the subject in response to treatment. In some cases, the sequencing comprises a method selected from one or more of sequencing by synthesis, sequencing by ligation, nanopore sequencing, nanoball sequencing, ion detection, sequencing by hybridization, polymerized colony (POLONY) sequencing, nanogrid rolling circle sequencing (ROLONY), and ion torrent sequencing. In some cases, the cell-free biological sample comprises less than 75 nanograms of nucleic acids. In some cases, the cell-free biological sample comprises a bodily fluid. In some cases, the bodily fluid is urine, saliva, blood, serum, plasma, tears, sputum, cerebrospinal fluid, synovial fluid, mucus, bile, semen, lymph, amniotic fluid, menstrual fluid, or combinations thereof. In some cases, the method further comprises computer processing the sequence to identify an epigenetic modification in the sequence. In some cases, the epigenetic modification is selected from methylation, phosphorylation, ubiquitination, sumoylation, acetylation, ribosylation, citrullination, and fragmentation. In some cases, the disease is cancer. In some cases, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, brain cancer, leukemia, lymphoma, and myeloma. In some cases, the method further comprises using at least the subset of the plurality sequence reads identified in processing to output an electronic report indicating that the subject has or is at risk of having the disease. In some cases, the method further comprises using at least the subset of the plurality sequence reads identified in the processing step to provide a therapeutic intervention to the subject for the disease. In some cases, the method further comprises using at least the subset of the plurality sequence reads identified in (d) to treat the subject for the disease. In some cases, the subject is treated by administering a chemotherapy or immunotherapy to the subject. In some cases, the disease is cancer. In some cases, the method further comprises using at least the subset of the plurality sequence reads identified in the processing step to monitor the subject for a progression or regression of the subject. In some cases, the processing operation comprises determining that at least 0.1% of the plurality of sequence reads corresponds to the plurality of reference sequence reads.

In one aspect, a method for determining that a subject has or is at risk of having a disease, may comprise obtaining a cell-free biological sample of the subject and using a plurality of nucleic acid molecules derived from the cell-free biological sample to generate a plurality of circular nucleic acid molecules. The method may comprise sequencing the plurality of circular nucleic acid molecules or derivatives thereof to sequencing to identify a plurality of breakpoint sequences and processing the plurality of breakpoint sequences against a plurality of reference breakpoint sequences to identify at least a subset of the plurality breakpoint sequences as corresponding to at least a subset of the plurality of reference breakpoint sequences, thereby determining that the subject has or is at risk of having the disease. In some cases, each of the plurality of breakpoint sequences comprises a 5′ end and a 3′ end of a nucleic acid molecule derived from the cell-free biological sample. In some cases, generating a plurality of circular nucleic acid molecules comprises ligating ends of each of the plurality of nucleic acid molecules to one another. In some cases, generating a plurality of circular nucleic acid molecules comprises coupling an adapter to a 3′ end, a 5′ end, or both a 5′end and a 3′end of each of the plurality of nucleic acid molecules. In some cases, the method further comprises subjecting each of the plurality of circular nucleic acid molecules to nucleic acid amplification to generate a plurality of amplification products of the plurality of circular nucleic acid molecules, wherein sequencing comprises subjecting the plurality of amplification products or derivatives thereof to sequencing to generate a plurality of breakpoint sequences of the plurality of amplification products or derivatives thereof. In some cases, the nucleic acid amplification is effected by a polymerase having strand-displacement activity. In some cases, the nucleic acid amplification is effected by a polymerase that does not have strand-displacement activity. In some cases, the nucleic acid amplification comprises contacting the circular nucleic acid molecule to an amplification reaction mixture comprising random primers. In some cases, the nucleic acid amplification comprises contacting the circular nucleic acid molecule to an amplification reaction mixture comprising one or more primers, each of which specifically hybridizes to a different target sequence via sequence complementarity. In some cases, the plurality of nucleic acid molecules is single stranded. In some cases, the plurality of nucleic acid molecules comprises cell-free DNA or RNA. In some cases, the plurality of nucleic acid molecules is from a tumor. In some cases, the method further comprises processing the sequence to monitor a progression or regression of the disease in the subject in response to treatment. In some cases, the sequencing comprises a method selected from one or more of sequencing by synthesis, sequencing by ligation, nanopore sequencing, nanoball sequencing, ion detection, sequencing by hybridization, polymerized colony (POLONY) sequencing, nanogrid rolling circle sequencing (ROLONY), and ion torrent sequencing. In some cases, the cell-free biological sample comprises less than 75 nanograms of nucleic acids. In some cases, the cell-free biological sample comprises a bodily fluid. In some cases, the bodily fluid is urine, saliva, blood, serum, plasma, tears, sputum, cerebrospinal fluid, synovial fluid, mucus, bile, semen, lymph, amniotic fluid, menstrual fluid, or combinations thereof. In some cases, the method further comprises computer processing the sequence to identify an epigenetic modification in the sequence. In some cases, the epigenetic modification is selected from methylation, phosphorylation, ubiquitination, sumoylation, acetylation, ribosylation, citrullination, and fragmentation. In some cases, the disease is cancer. In some cases, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, brain cancer, leukemia, lymphoma, and myeloma. In some cases, the method further comprises using at least the subset of the plurality breakpoint sequences identified in (d) to output an electronic report indicating that the subject has or is at risk of having the disease. In some cases, the method further comprises using at least the subset of the plurality breakpoint sequences identified in (d) to provide a therapeutic intervention to the subject for the disease. In some cases, the method further comprises using at least the subset of the plurality breakpoint sequences identified in (d) to treat the subject for the disease. In some cases, the subject is treated by administering a chemotherapy or immunotherapy to the subject. In some cases, the disease is cancer. In some cases, the method further comprises using at least the subset of the plurality breakpoint sequences identified in (d) to monitor the subject for a progression or regression of the subject. In some cases, the method further comprises determining that at least 0.1% of the plurality of breakpoint sequences corresponds to the plurality of reference breakpoint sequences.

In one aspect, a method for determining that a subject has or is at risk of having a disease, may comprise accessing a first database comprising a plurality of sequence reads, wherein a sequence read of the plurality of sequence reads comprises multiple copies of a sequence of a nucleic acid molecule from the subject, and a second database comprising a plurality of reference sequence reads, and processing the plurality of sequence reads from the first database against the plurality of reference sequence reads from the second database to identify at least a subset of the plurality sequence reads as corresponding to at least a subset of the plurality of reference sequence reads, thereby determining that the subject has or is at risk of having the disease. In some cases, the first database and the second database are different databases. In some cases, the disease is cancer.

In one aspect, a method for determining that a subject has or is at risk of having a disease, may comprise accessing a first database comprising a plurality of breakpoint sequences, wherein a breakpoint sequence of the plurality of breakpoint sequences comprises multiple copies of a sequence of a 5′ end and a 3′ end of a nucleic acid molecule of the subject, and a second database comprising a plurality of reference breakpoint sequences and processing the plurality of breakpoint sequences from the first database against the plurality of reference breakpoint sequences from the second database to identify at least a subset of the plurality breakpoint sequences as corresponding to at least a subset of the plurality of reference breakpoint sequences, thereby determining that the subject has or is at risk of having the disease. In some cases, the first database and the second database are different databases. In some cases, the disease is cancer.

In one aspect, a system for determining that a subject has or is at risk of having a disease may comprise a first database comprising a plurality of sequence reads, wherein a sequence read of the plurality of sequence reads comprises multiple copies of a sequence of a nucleic acid molecule of the subject; a second database comprising a plurality of reference sequence reads; and one or more computer processors that are individually or collectively programed to process the plurality of sequence reads from the first database against the plurality of reference sequence reads from the second database to identify at least a subset of the plurality sequence reads as corresponding to at least a subset of the plurality of reference sequence reads, thereby determining that the subject has or is at risk of having the disease. In some cases, the first database and the second database are different databases. In some cases, the disease is cancer.

In one aspect, a system for determining that a subject has or is at risk of having a disease, may comprise a first database comprising a plurality of breakpoint sequences, wherein a breakpoint sequence of the plurality of breakpoint sequences comprises multiple copies of a sequence of a 5′ end and a 3′ end of a nucleic acid molecule of the subject; a second database comprising a plurality of reference breakpoint sequences; and one or more computer processors that are individually or collectively programed to process the plurality of breakpoint sequences from the first database against the plurality of reference breakpoint sequences from the second database to identify at least a subset of the plurality breakpoint sequences as corresponding to at least a subset of the plurality of reference breakpoint sequences, thereby determining that the subject has or is at risk of having the disease. In some cases, the first database and the second database are different databases. In some cases, the disease is cancer.

In one aspect, a non-transitory computer-readable medium may comprise machine-executable code that, upon execution by one or more computer processors, implements a method for determining that a subject has or is at risk of having a disease, comprising accessing a first database comprising a plurality of sequence reads, wherein a sequence read of the plurality of sequence reads comprises multiple copies of a sequence of a nucleic acid molecule from the subject; and a second database comprising a plurality of reference sequence reads; and processing the plurality of sequence reads from the first database against the plurality of reference sequence reads from the second database to identify at least a subset of the plurality sequence reads as corresponding to at least a subset of the plurality of reference sequence reads, thereby determining that the subject has or is at risk of having the disease. In some cases, the first database and the second database are different databases. In some cases, the disease is cancer.

In one aspect, a non-transitory computer-readable medium comprising machine-executable code that, upon execution by one or more computer processors, implements a method for determining that a subject has or is at risk of having a disease may comprise accessing a first database comprising a plurality of breakpoint sequences, wherein a breakpoint sequence of the plurality of breakpoint sequences comprises multiple copies of a sequence of a 5′ end and a 3′ end of a nucleic acid molecule of the subject; and a second database comprising a plurality of reference breakpoint sequences; and processing the plurality of breakpoint sequences from the first database against the plurality of reference breakpoint sequences from the second database to identify at least a subset of the plurality breakpoint sequences as corresponding to at least a subset of the plurality of reference breakpoint sequences, thereby determining that the subject has or is at risk of having the disease. In some cases, the first database and the second database are different databases. In some cases, the disease is cancer.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example method for analyzing cell-free DNA in a subject to determine that a subject has or is at risk of having a disease, such as cancer.

FIG. 2 illustrates an example computer system.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein the term “about” or “approximately” means within an acceptable error range for the particular value as determined by one of ordinary skill in the art, which may depend in part on how the value is measured or determined, i.e., the limitations of the measurement system. For example, “about” can mean within 1 or more than 1 standard deviation, per the practice in the art. As another example, “about” can mean a range of up to 20%, up to 10%, up to 5%, or up to 1% of a given value. With respect to biological systems or processes, the term “about” can mean within an order of magnitude, such as within 5-fold or within 2-fold of a value. Where particular values are described in the application and claims, unless otherwise stated, the term “about” means within an acceptable error range for the particular value.

As used herein, the terms “polynucleotide”, “nucleotide”, “nucleotide sequence”, “nucleic acid” and “oligonucleotide” are used interchangeably and generally refer to a polymeric form of nucleotides of any length, either deoxyribonucleotides (DNA) or ribonucleotides (RNA), or analogs thereof. Polynucleotides may have any three dimensional structure, and may perform any function. The following are non-limiting examples of polynucleotides: cell-free nucleic acids, cell-free DNA (cfDNA), cell-free RNA (cfRNA), circulating tumor DNA (ctDNA), circulating tumor RNA (ctRNA), coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “subject,” as used herein, generally refers to a vertebrate, such as a mammal (e.g., a human). Mammals include, but are not limited to, murines, simians, humans, farm animals, sport animals, and pets (e.g., a dog or a cat). Tissues, cells, and their progeny of a biological entity obtained in vivo or cultured in vitro are also encompassed. The subject may be a patient. The subject may be symptomatic with respect to a disease (e.g., cancer). Alternatively, the subject may be asymptomatic with respect to the disease.

The term “biological sample,” as used herein, generally refers to a sample derived from or obtained from a subject, such as a mammal (e.g., a human). Biological samples may include, but are not limited to, hair, finger nails, skin, sweat, tears, ocular fluids, nasal swab or nasopharyngeal wash, sputum, throat swab, saliva, mucus, blood, serum, plasma, placental fluid, amniotic fluid, cord blood, emphatic fluids, cavity fluids, earwax, oil, glandular secretions, bile, lymph, pus, microbiota, meconium, breast milk, bone marrow, bone, CNS tissue, cerebrospinal fluid, adipose tissue, synovial fluid, stool, gastric fluid, urine, semen, vaginal secretions, stomach, small intestine, large intestine, rectum, pancreas, liver, kidney, bladder, lung, and other tissues and fluids derived from or obtained from a subject. The biological sample may be a cell-free (or cell free) biological sample.

The term “cell-free biological sample,” as used herein, generally refers to a sample derived from or obtained from a subject that is free from cells. Cell-free biological samples may include, but are not limited to, blood, serum, plasma, nasal swab or nasopharyngeal wash, saliva, urine, gastric fluid, tears, stool, mucus, sweat, earwax, oil, glandular secretion, bile, lymph, cerebrospinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions.

The term “reference sequence reads,” as used herein, generally refers to sequence reads identified as being significant to or associated with a disease, such as cancer. Such reference sequence reads may be generated from known samples (e.g., samples from subject with a known disease, such as cancer).

The term “breakpoint sequences,” as used herein, generally refers to sequences found at the ends of nucleic acid fragments, such as sequences found at the end of cfDNA fragments. A breakpoint sequence may comprise a 5′ end and a 3′ end of a nucleic acid molecule, such as a nucleic acid molecule derived from the cell-free biological sample. A breakpoint sequence may be referred to as a junction sequence. A breakpoint sequence may be also be referred to as a fusion sequence.

The term “reference breakpoint sequences,” as used herein, generally refers to breakpoint sequences identified as being significant to or associated with a disease, such as cancer. Such reference breakpoint sequences may be generated from known samples (e.g., samples from subject with a known disease, such as cancer). Reference breakpoint sequences may also be referred to as reference junction sequences or reference fusion sequences.

The terms “early stage cancer” and “non-metastatic cancer,” as used herein, generally refer to a cancer that has not yet metastasized in a subject (i.e., the cancer has not left its initial location to spread to other locations). The exact staging may depend upon the type of cancer, details for which are provided elsewhere herein.

The terms “tumor burden” and “tumor load,” as used herein, generally refer to the size of a tumor or the amount of cancer in the body of the subject.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Provided herein are methods, systems, and compositions for determining whether a subject has a disease (e.g., cancer) or is at risk of having a disease (e.g., cancer) based on identifying from a cell-free biological sample a subset of sequence reads that correspond to a reference sequence read and/or breakpoint sequences that correspond to a reference breakpoint sequence thereby determining that the subject has or is at risk of having a disease, such as cancer.

### Use of Sequence Reads for Detecting a Disease

Provided herein are methods for determining whether a subject has or is at risk of having a disease (e.g., cancer) based at least in part on sequence reads. A method for determining whether a subject has or is at risk of having the disease (e.g., cancer) may comprise obtaining a cell-free biological sample of the subject and using a plurality of nucleic acid molecules derived from the cell-free biological sample to generate a plurality of circular nucleic acid molecules. Next, the plurality of circular nucleic acid molecules or derivatives thereof may be subject to sequencing to generate a plurality of sequence reads. The plurality of sequence reads may be processed against a plurality of reference sequence reads to identify at least a subset of the plurality sequence reads as corresponding to at least a subset of the plurality of reference sequence reads, thereby determining that the subject has or is at risk of having the disease, such as cancer.

In a method of determining that a subject has or is at risk having of a disease (e.g., cancer), a plurality of circular nucleic acid molecules may be generated by ligating ends of each of the plurality of nucleic acid molecules to one another. Alternatively or in combination, a plurality of circular nucleic acid molecules may be generated by coupling an adapter to a 3′ end, a 5′ end, or both a 5′end and a 3′end of each of the plurality of nucleic acid molecules.

In some cases, subsequent to a circularization operation, each of the plurality of circular nucleic acid molecules may be subjected to nucleic acid amplification to generate a plurality of amplification products of the plurality of circular nucleic acid molecules, wherein a sequencing step comprises subjecting the plurality of amplification products or derivatives thereof to sequencing to generate a plurality of sequence reads of the plurality of amplification products or derivatives thereof The nucleic acid amplification may be effected by a polymerase having strand-displacement activity. Polymerases having strand-displacement activity may include, but are not limited to, Bst DNA polymerase (large fragment), Deep Vent DNA polymerase, Klenow Fragment DNA polymerase, M-MuLV Reverse Transcriptase, phi29 DNA polymerase, Vent_{R} DNA polymerase, and other polymerases known to have strand displacement activity. Alternatively, the nucleic acid amplification may be effected by a polymerase that does not have strand-displacement activity. Polymerases not having strand-displacement activity may include, but are not limited to, Bst DNA polymerase (full length), Crimson Taq DNA polymerase, E. coli DNA polymerase I, LongAmp Taq DNA polymerase, OneTaq DNA polymerase, Phusion High-Fidelity DNA polymerase, Q5 DNA polymerase, Sulfolobus DNA polymerase IV, T4 DNA polymerase, T7 DNA polymerase Taq DNA polymerase, and other polymerases known to have no strand displacement activity. In some cases, the nucleic acid amplification comprises contacting the circular nucleic acid molecule to an amplification reaction mixture comprising random primers. Alternatively, the nucleic acid amplification comprises contacting the circular nucleic acid molecule to an amplification reaction mixture comprising one or more primers, each of which specifically hybridizes to a different target sequence via sequence complementarity.

Methods provided herein may utilize a plurality of nucleic acid molecules derived from a cell-free biological sample. In some cases, the plurality of nucleic acid molecules is single stranded. Alternatively or in combination, the plurality of nucleic acid molecules is double stranded. In some cases, the plurality of nucleic acid molecules comprises cell-free DNA. Alternatively or in combination, the plurality of nucleic acid molecules comprises cell-free RNA. The plurality of nucleic acid molecules may be from a tumor.

Methods of determining that a subject has or is at increased risk of having (or developing) a disease (e.g., cancer) may further comprise processing the plurality of sequence reads to monitor a progression of a disease (e.g., cancer) in the subject in response to treatment.

Sequencing may comprise sequencing by synthesis (e.g., Illumina, Pacific Biosciences of California, or Ion Torrent), sequencing by ligation, nanopore sequencing (e.g., Oxford Nanopore), nanoball sequencing, ion detection, sequencing by hybridization, polymerized colony (POLONY) sequencing, and nanogrid rolling circle sequencing (ROLONY).

In methods provided herein, cell-free biological samples may comprise small amounts of nucleic acids. In some cases, cell-free biological samples herein comprise less than 1000 nanograms, less than 900 nanograms, less than 800 nanograms, less than 700 nanograms, less than 600 nanograms, less than 500 nanograms, less than 400 nanograms, less than 300 nanograms, less than 200 nanograms, less than 100 nanograms, less than 75 nanograms, less than 50 nanograms, or less than 25 nanograms of nucleic acids. In some cases, cell-free biological samples herein comprise at least 1000 nanograms, at least 900 nanograms, at least 800 nanograms, at least 700 nanograms, at least 600 nanograms, at least 500 nanograms, at least 400 nanograms, at least 300 nanograms, at least 200 nanograms, at least 100 nanograms, at least 75 nanograms, at least 50 nanograms, or at least 25 nanograms of nucleic acids.

Biological samples that are free or substantially free of cells may be used. In some cases, cell-free biological samples may comprise a bodily fluid. Cell-free biological samples or bodily fluids may include, but are not limited to, urine, saliva, blood, serum, plasma, tears, sputum, cerebrospinal fluid, synovial fluid, mucus, bile, semen, lymph, amniotic fluid, menstrual fluid, or combinations thereof. Additional suitable cell-free biological samples or bodily fluids may be used in methods herein as described herein. In some cases, cell-free biological samples have been processed to remove cells from the sample prior to use in methods herein.

In additional aspects of methods provided herein, methods may further comprise computer processing the plurality of sequence reads to identify an epigenetic modification in the plurality of sequence reads. Epigenetic modifications herein generally refer to heritable alterations may not be due to changes in a DNA sequence and may have been observed to regulate patterns of gene expression. In some cases, epigenetic modifications may include but are not limited to methylation, phosphorylation, ubiquitination, sumoylation, acetylation, ribosylation, citrullination, fragmentation, and combinations thereof

Methods herein may identify a subject as having or being at risk of having (or developing) a disease, such as, for example, cancer. Such risk may be an increased risk relative to a control. Examples of cancer include, but are not limited to, colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, brain cancer, leukemia, lymphoma, and myeloma.

Methods provided herein may comprise additional operations including, but not limited to, output of electronic reports and administering treatment to a subject. In some cases methods herein further comprise using at least the subset of the plurality sequence reads identified in the processing step to output an electronic report indicating that the subject has or is at risk of having the disease (e.g., cancer). In some cases, methods herein further comprise using at least the subset of the plurality sequence reads identified in the processing step to provide a therapeutic intervention to the subject for the disease (e.g., cancer). In some cases, methods herein further comprise using at least the subset of the plurality sequence reads identified in the processing step to treat the subject for the disease (e.g., cancer). In some cases, methods herein further comprise treating the subject by administering a chemotherapy or immunotherapy to the subject. In some cases, methods herein further comprise using at least the subset of the plurality sequence reads identified in the processing step to monitor the subject for a progression or regression of the disease (e.g., cancer) in the subject.

Methods of determining that a subject has or has increased risk of having a disease (e.g., cancer) herein comprise processing a plurality of sequence reads against a plurality of reference sequence reads, thereby identifying at least a subset of the plurality of sequence reads as corresponding to at least a subset of the plurality of reference sequence reads. Such processing may include comparing a sequence read to a reference sequence read. Such processing may not include comparing the sequence read to a consensus sequence.

In some embodiments, the processing comprises determining that at least 80%, 85%, 90%, 95%, or more of the plurality of sequence reads corresponds to the plurality of reference sequence reads. In some embodiments, the processing comprises determining that at least 0.01% of the plurality of sequence reads corresponds to the plurality of reference sequence reads. In some embodiments, the processing comprises determining that at least 0.1% of the plurality of sequence reads corresponds to the plurality of reference sequence reads. In some embodiments, the processing comprises determining that at least 1% of the plurality of sequence reads corresponds to the plurality of reference sequence reads. In some embodiments, the processing comprises determining that at least 5% of the plurality of sequence reads corresponds to the plurality of reference sequence reads. In some embodiments, the processing comprises determining that at least 10% of the plurality of sequence reads corresponds to the plurality of reference sequence reads. In some embodiments, the processing comprises determining that at least 20% of the plurality of sequence reads corresponds to the plurality of reference sequence reads. In some embodiments, the processing comprises determining that at least 30% of the plurality of sequence reads corresponds to the plurality of reference sequence reads. In some embodiments, the processing comprises determining that at least 40% of the plurality of sequence reads corresponds to the plurality of reference sequence reads. In some embodiments, the processing comprises determining that at least 50% of the plurality of sequence reads corresponds to the plurality of reference sequence reads. In some embodiments, the processing comprises determining that at least 60% of the plurality of sequence reads corresponds to the plurality of reference sequence reads. In some embodiments, the processing comprises determining that at least 70% of the plurality of sequence reads corresponds to the plurality of reference sequence reads. In some embodiments, the processing comprises determining that at least 80%, 85%, 90%, 95%, or more of the plurality of sequence reads corresponds to the plurality of reference sequence reads.

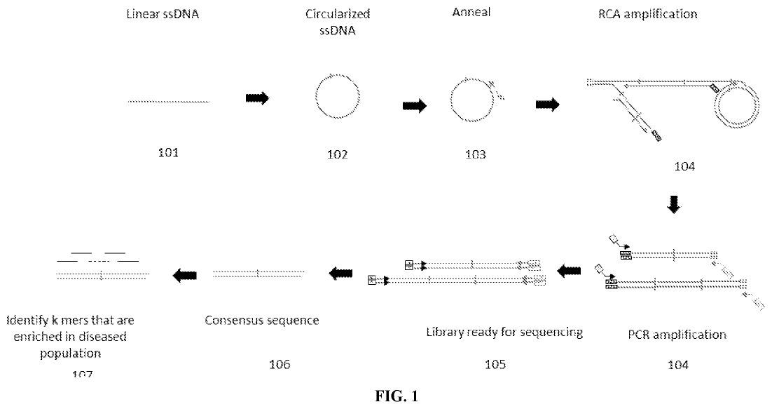

FIG. 1 illustrates an example method of detecting a disease, such as cancer. The example method begins with a linear single stranded DNA molecule (ssDNA) 101, which is subjected to circularization resulting in a circularized ssDNA molecule 102. A primer is annealed to the ssDNA molecule 103 and the circularized ssDNA molecule is subjected to rolling circle amplification (RCA) 104. The products of RCA amplification are subjected to PCR amplification 104 to produce a library that is ready for sequencing 105. The sequencing reads are processed to result in a consensus sequence 106. This method is performed on a plurality of ssDNA molecules present in a biological sample, allowing one to identify kmers that are enriched in subjects with a disease 107, such as cancer.

### Use of Breakpoint Sequences for Detecting a Disease

Provided herein are methods for determining whether a subject has or is at risk of having a disease (e.g., cancer) based least in part on breakpoint sequences. A method for determining whether a subject has or is at risk of having the disease (e.g., cancer) may comprise obtaining a cell-free biological sample of the subject and using a plurality of nucleic acid molecules derived from the cell-free biological sample to generate a plurality of circular nucleic acid molecules. Next, the plurality of circular nucleic acid molecules or derivatives thereof may be subjected to sequencing to identify a plurality of breakpoint sequences. The plurality of breakpoint sequences may be processed against a plurality of reference breakpoint sequences to identify at least a subset of the plurality breakpoint sequences as corresponding to at least a subset of the plurality of reference breakpoint sequences, thereby determining that the subject has or is at increased risk of having the disease, such as cancer. Each or at least a subset of the plurality of breakpoint sequences may comprise a 5′ end and a 3′ end of a nucleic acid molecule derived from the cell-free biological sample.

In a method of determining that a subject has or is at increased risk of having a disease, such as cancer herein, in some cases, a plurality of circular nucleic acid molecules may be generated by ligating ends of each of the plurality of nucleic acid molecules to one another. Alternatively or in combination, a plurality of circular nucleic acid molecules may be generated by coupling an adapter to a 3′ end, a 5′ end, or both a 5′end and a 3′end of each of the plurality of nucleic acid molecules.

In some cases, methods herein further comprise, subsequent to a circularization step, each of the plurality of circular nucleic acid molecules may be amplified to generate a plurality of amplification products of the plurality of circular nucleic acid molecules, wherein a sequencing step comprises sequencing the plurality of amplification products or derivatives thereof to generate a plurality of breakpoint sequences of the plurality of amplification products or derivatives thereof. Nucleic acid amplification may be effected by a polymerase having strand-displacement activity. Polymerases having strand-displacement activity may include, but are not limited to, Bst DNA polymerase (large fragment), Deep Vent DNA polymerase, Klenow Fragment DNA polymerase, M-MuLV Reverse Transcriptase, phi29 DNA polymerase, Vent_{R} DNA polymerase, and other polymerases known to have strand displacement activity. Alternatively, nucleic acid amplification may be effected by a polymerase that does not have strand-displacement activity. Polymerases that do not have strand-displacement activity may include, but are not limited to, Bst DNA polymerase (full length), Crimson Taq DNA polymerase, E. coli DNA polymerase I, LongAmp Taq DNA polymerase, OneTaq DNA polymerase, Phusion High-Fidelity DNA polymerase, Q5 DNA polymerase, Sulfolobus DNA polymerase IV, T4 DNA polymerase, T7 DNA polymerase Taq DNA polymerase, and other polymerases known to have no strand displacement activity. In some cases, the nucleic acid amplification comprises contacting the circular nucleic acid molecule to an amplification reaction mixture comprising random primers. Alternatively, the nucleic acid amplification comprises contacting the circular nucleic acid molecule to an amplification reaction mixture comprising one or more primers, each of which specifically hybridizes to a different target sequence via sequence complementarity.

Methods provided herein utilize a plurality of nucleic acid molecules derived from a cell-free biological sample. In some cases, the plurality of nucleic acid molecules is single stranded. Alternatively or in combination, the plurality of nucleic acid molecules is double stranded. In some cases, the plurality of nucleic acid molecules comprises cell-free DNA. Alternatively or in combination, the plurality of nucleic acid molecules comprises cell-free RNA. The plurality of nucleic acid molecules may be from a tumor.

Methods of determining that a subject has or is at increased risk of having (or developing) a disease, such as cancer, may further comprise processing the plurality of breakpoint sequences to monitor a progression of a disease, such as cancer, in the subject in response to treatment.

In methods provided herein, the sequencing may comprise a method including, but not limited to, sequencing by synthesis, sequencing by ligation, nanopore sequencing, nanoball sequencing, ion detection, sequencing by hybridization, polymerized colony (POLONY) sequencing, nanogrid rolling circle sequencing (ROLONY), ion torrent sequencing, and other appropriate sequencing methods.

In methods provided herein, the cell-free biological samples may comprise small amounts of nucleic acids. In some cases, cell-free biological samples herein comprise less than 1000 nanograms, less than 900 nanograms, less than 800 nanograms, less than 700 nanograms, less than 600 nanograms, less than 500 nanograms, less than 400 nanograms, less than 300 nanograms, less than 200 nanograms, less than 100 nanograms, less than 75 nanograms, less than 50 nanograms, or less than 25 nanograms of nucleic acids. In some cases, cell-free biological samples herein comprise at least 1000 nanograms, at least 900 nanograms, at least 800 nanograms, at least 700 nanograms, at least 600 nanograms, at least 500 nanograms, at least 400 nanograms, at least 300 nanograms, at least 200 nanograms, at least 100 nanograms, at least 75 nanograms, at least 50 nanograms, or at least 25 nanograms of nucleic acids.

In methods provided herein, biological samples may be used that are free of cells, for example, the cell-free biological sample may comprise a bodily fluid. The cell-free biological samples or bodily fluids may include, but are not limited to, urine, saliva, blood, serum, plasma, tears, sputum, cerebrospinal fluid, synovial fluid, mucus, bile, semen, lymph, amniotic fluid, menstrual fluid, or combinations thereof. Additional suitable cell-free biological samples or bodily fluids may be used in methods herein described herein. In some cases, cell-free biological samples have been processed to remove cells from the sample prior to use in methods herein.

In additional aspects of methods provided herein, methods further comprise computer processing the breakpoint sequences to identify an epigenetic modification in the sequence. Epigenetic modifications herein refer to heritable alterations that are not due to changes in DNA sequence and have often been observed to regulate patterns of gene expression. Epigenetic modifications may include, but are not limited to, methylation, phosphorylation, ubiquitination, sumoylation, acetylation, ribosylation, citrullination, fragmentation, and combinations thereof.

Methods herein identify a subject as having or having increased risk of developing a disease, such as, for example, cancer. Examples of cancer include, but are not limited to, colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, brain cancer, leukemia, lymphoma, and myeloma.

Methods provided herein often comprise additional steps including but not limited to output of electronic reports and administering treatment to a subject. In some cases, methods herein further comprise using at least the subset of the plurality breakpoint sequences identified in the processing step to output an electronic report indicating that the subject has or is at risk of having the disease (e.g., cancer). In some cases, methods herein further comprise using at least the subset of the plurality breakpoint sequences identified in the processing step to provide a therapeutic intervention to the subject for the disease (e.g., cancer). In some cases, methods herein further comprise using at least the subset of the plurality breakpoint sequences identified in the processing step to treat the subject for the disease (e.g., cancer). In some cases, methods herein further comprise treating the subject by administering a chemotherapy or immunotherapy to the subject. In some cases, methods herein further comprise using at least the subset of the plurality breakpoint sequences identified in the processing step to monitor the subject for a progression or regression of the subject.

Methods of determining that a subject as has or is at increased risk of having a disease, such as cancer, herein comprise processing a plurality of breakpoint sequences against a plurality of reference breakpoint sequences, thereby identifying at least a subset of the plurality of breakpoint sequences as corresponding to at least a subset of the plurality of reference breakpoint sequences. In some embodiments, the processing comprises determining that at least 80%, 85%, 90%, 95%, or more of the plurality of breakpoint sequences corresponds to the plurality of reference breakpoint sequences. In some embodiments, the processing comprises determining that at least 0.01% of the plurality of breakpoint sequences corresponds to the plurality of reference breakpoint sequences. In some embodiments, the processing comprises determining that at least 0.1% of the plurality of breakpoint sequences corresponds to the plurality of reference breakpoint sequences. In some embodiments, the processing comprises determining that at least 1% of the plurality of breakpoint sequences corresponds to the plurality of reference breakpoint sequences. In some embodiments, the processing comprises determining that at least 5% of the plurality of breakpoint sequences corresponds to the plurality of reference breakpoint sequences. In some embodiments, the processing comprises determining that at least 10% of the plurality of breakpoint sequences corresponds to the plurality of reference breakpoint sequences. In some embodiments, the processing comprises determining that at least 20% of the plurality of breakpoint sequences corresponds to the plurality of reference breakpoint sequences. In some embodiments, the processing comprises determining that at least 30% of the plurality of breakpoint sequences corresponds to the plurality of reference breakpoint sequences. In some embodiments, the processing comprises determining that at least 40% of the plurality of breakpoint sequences corresponds to the plurality of reference breakpoint sequences. In some embodiments, the processing comprises determining that at least 50% of the plurality of breakpoint sequences corresponds to the plurality of reference breakpoint sequences. In some embodiments, the processing comprises determining that at least 60% of the plurality of breakpoint sequences corresponds to the plurality of reference breakpoint sequences. In some embodiments, the processing comprises determining that at least 70% of the plurality of breakpoint sequences corresponds to the plurality of reference breakpoint sequences. In some embodiments, the processing comprises determining that at least 80%, 85%, 90%, 95%, or more of the plurality of breakpoint sequences corresponds to the plurality of reference breakpoint sequences.

### Systems and Computer Assisted Methods

Provided herein are systems for determining that a subject has or is at risk of having a disease (e.g., cancer). A system for determining whether a subject has or is at risk of having the disease (e.g., cancer) may comprise: a first database comprising a plurality of sequence reads, wherein a sequence read of the plurality of sequence reads may comprise multiple copies of a sequence of a nucleic acid molecule of the subject and a second database comprising a plurality of reference sequence reads. The system may comprise one or more computer processors that are individually or collectively programed to process the plurality of sequence reads from the first database against the plurality of reference sequence reads from the second database to identify at least a subset of the plurality sequence reads as corresponding to at least a subset of the plurality of reference sequence reads, thereby determining that the subject has or is at risk of having the disease (e.g., cancer). In some embodiments, the first database and the second database are different databases.

A system for determining that a subject has or is at risk of having a disease (e.g., cancer) may also comprise a first database comprising a plurality of breakpoint sequences, wherein a breakpoint sequence of the plurality of breakpoint sequences may comprises multiple copies of a sequence of a 5′ end and a 3′ end of a nucleic acid molecule of the subject and a second database comprising a plurality of reference breakpoint sequences. The system may comprise one or more computer processors that are individually or collectively programed to process the plurality of breakpoint sequences from the first database against the plurality of reference breakpoint sequences from the second database to identify at least a subset of the plurality breakpoint sequences as corresponding to at least a subset of the plurality of reference breakpoint sequences, thereby determining that the subject has or is at risk of having the disease (e.g., cancer). In some cases, the first database and the second database are different databases.

Further provided herein are non-transitory computer-readable mediums. A non-transitory computer-readable mediums may comprise machine-executable code that, upon execution by one or more computer processors, implements a method for determining that a subject has or is at risk of having a disease (e.g., cancer) comprising accessing a first database comprising a plurality of sequence reads, wherein a sequence read of the plurality of sequence reads comprises multiple copies of a sequence of a nucleic acid molecule from the subject and accessing a second database comprising a plurality of reference sequence reads. The method may comprise processing the plurality of sequence reads from the first database against the plurality of reference sequence reads from the second database to identify at least a subset of the plurality sequence reads as corresponding to at least a subset of the plurality of reference sequence reads, thereby determining that the subject has or is at risk of having the disease (e.g., cancer). In some embodiments, the first database and the second database are different databases.

Non-transitory computer-readable mediums may also comprise machine-executable code that, upon execution by one or more computer processors, implements a method for determining that a subject has or is at risk of having a disease, such as cancer, comprising accessing a first database comprising a plurality of breakpoint sequences, wherein a breakpoint sequence of the plurality of breakpoint sequences comprises multiple copies of a sequence of a 5′ end and a 3′ end of a nucleic acid molecule of the subject and accessing a second database comprising a plurality of reference breakpoint sequences. The method may comprise processing the plurality of breakpoint sequences from the first database against the plurality of reference breakpoint sequences from the second database to identify at least a subset of the plurality breakpoint sequences as corresponding to at least a subset of the plurality of reference breakpoint sequences, thereby determining that the subject has or is at risk of having the disease (e.g., cancer). In some embodiments, the first database and the second database are different databases.

A computer for use in the system can comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A client-server, relational database architecture can be used in embodiments of the system. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments, the server computer handles all of the database functionality. The client computer can have software that handles all the front-end data management and can also receive data input from users.

The system can be configured to receive a user request to perform a detection reaction on a sample. The user request may be direct or indirect. Examples of direct request include those transmitted by way of an input device, such as a keyboard, mouse, or touch screen. Examples of indirect requests include transmission via a communication medium, such as over the internet (either wired or wireless).

The system can further comprise an amplification system that performs a nucleic acid amplification reaction on the sample or a portion thereof in response to the user request. A variety of methods of amplifying polynucleotides (e.g. DNA and/or RNA) are available. Amplification may be linear, exponential, or involve both linear and exponential phases in a multi-phase amplification process. Amplification methods may involve changes in temperature, such as a heat denaturation step, or may be isothermal processes that do not require heat denaturation. Non-limiting examples of suitable amplification processes are described herein, such as with regard to any of the various aspects of the disclosure. In some embodiments, amplification comprises rolling circle amplification (RCA). A variety of systems for amplifying polynucleotides are available, and may vary based on the type of amplification reaction to be performed. For example, for amplification methods that comprise cycles of temperature changes, the amplification system may comprise a thermocycler. An amplification system can comprise a real-time amplification and detection instrument, such as systems manufactured by Applied Biosystems, Roche, and Strategene. In some embodiments, the amplification reaction comprises the steps of (i) circularizing individual polynucleotides to form a plurality of circular polynucleotides, each of which having a junction between the 5′ end and 3′ end; and (ii) amplifying the circular polynucleotides. Samples, polynucleotides, primers, polymerases, and other reagents can be any of those described herein, such as with regard to any of the various aspects. Non-limiting examples of circularization processes (e.g. with and without adapter oligonucleotides), reagents (e.g. types of adapters, use of ligases), reaction conditions (e.g. favoring self-joining), optional additional processing (e.g. post-reaction purification), and the junctions formed thereby are provided herein, such as with regard to any of the various aspects of the disclosure. Systems can be selected and or designed to execute any such methods.

Systems may further comprise a sequencing system that generates sequencing reads for polynucleotides amplified by the amplification system, identifies sequence differences between sequencing reads and a reference sequence, and calls a sequence difference that occurs in at least two circular polynucleotides having different junctions as the sequence variant. The sequencing system and the amplification system may be the same, or comprise overlapping equipment. For example, both the amplification system and sequencing system may utilize the same thermocycler. A variety of sequencing platforms for use in the system are available, and may be selected based on the selected sequencing method. Examples of sequencing methods are described herein. Amplification and sequencing may involve the use of liquid handlers. Several commercially available liquid handling systems can be utilized to run the automation of these processes (see for example liquid handlers from Perkin-Elmer, Beckman Coulter, Caliper Life Sciences, Tecan, Eppendorf, Apricot Design, Velocity 11 as examples). A variety of automated sequencing machines are commercially available, and include sequencers manufactured by Life Technologies (SOLiD platform, and pH-based detection), Roche (454 platform), Illumina (e.g. flow cell based systems, such as Genome Analyzer devices). Transfer between 2, 3, 4, 5, or more automated devices (e.g. between one or more of a liquid handler and a sequencing device) may be manual or automated.

The system can further comprise a report generator that sends a report to a recipient, wherein the report contains results for detection of the sequence variant. A report may be generated in real-time, such as during a sequencing read or while sequencing data is being analyzed, with periodic updates as the process progresses. In addition, or alternatively, a report may be generated at the conclusion of the analysis. The report may be generated automatically, such when the sequencing system completes the step of calling all sequence variants. In some embodiments, the report is generated in response to instructions from a user. In addition to the results of detection of the sequence variant, a report may also contain an analysis based on the one or more sequence variants. For example, where one or more sequence variants are associated with a particular contaminant or phenotype, the report may include information concerning this association, such as a likelihood that the contaminant or phenotype is present, at what level, and optionally a suggestion based on this information (e.g. additional tests, monitoring, or remedial measures). The report can take any of a variety of forms. It is envisioned that data relating to the present disclosure can be transmitted over such networks or connections (or any other suitable approach for transmitting information, including but not limited to mailing a physical report, such as a print-out) for reception and/or for review by a receiver. The receiver can be but is not limited to an individual, or electronic system (e.g. one or more computers, and/or one or more servers).

A machine readable medium comprising computer-executable code may take many forms, including but not limited to, a tangible storage medium, a carrier wave medium or physical transmission medium. Non-volatile storage media include, for example, optical or magnetic disks, such as any of the storage devices in any computers) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device comprising a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch -sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Methods of Library Preparation and Amplification

Methods herein comprise, in certain cases, amplification of polynucleotides present in a sample from a subject. Methods of amplification used herein often comprise rolling-circle amplification. Alternatively or in combination, methods of amplification used herein comprise PCR. In some cases, methods of amplification herein comprise linear amplification. Often amplification is not targeted to one gene or set of genes and the entire nucleic acid sample is amplified. In some cases, the method comprises (a) circularizing individual polynucleotides of the plurality to form a plurality of circular polynucleotides, each of which having a junction between the 5′ end and the 3′ end; and (b) amplifying the circular polynucleotides of (a) to produce amplified polynucleotides. In additional cases, methods of amplification comprise (c) shearing the amplified polynucleotides to produce sheared polynucleotides, each sheared polynucleotide comprising one or more shear points at a 5′ end and/or 3′ end. In some cases, the method does not comprise enriching for a target sequence.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”).

Samples herein comprise polynucleotides having a mixture of ends formed by natural degradation processes (such as cell lysis, cell death, and other processes by which polynucleotides such as DNA and RNA are released from a cell to its surrounding environment in which it may be further degraded, e.g., cell-free polynucleotides, e.g., cell-free DNA and cell-free RNA), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as DNase I, fragmentase). Where samples comprise polynucleotides having a mixture of ends, the likelihood of two polynucleotides having the same 5′ end or 3′ end is low, and the likelihood that two polynucleotides will independently have both the same 5′ end and 3′ end is lower. Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

In some embodiments, circularizing individual polynucleotides in (a) is effected by subjected the plurality of polynucleotides to a ligation reaction. The ligation reaction may comprise a ligase enzyme. In some embodiments, the ligase enzyme is degraded prior to amplifying in (b). Degradation of ligase prior to amplifying in (b) can increase the recovery rate of amplifiable polynucleotides. In some embodiments, the plurality of circularized polynucleotides are not purified or isolated prior to (b). In some embodiments, uncircularized, linear polynucleotides are degraded prior to amplifying.

In some cases, circularizing in (a) comprises the step of joining and adapter polynucleotide to the 5′ end, the 3′ end, or both the 5′ end and the 3′ end of a polynucleotide in the plurality of polynucleotides. As previously described, where the 5′ end and/or 3′ end of a polynucleotide are joined via an adapter polynucleotide, the term “junction” can refer to the junction between the polynucleotide and the adapter (e.g., one of the 5′ end junction or the 3′ end junction), or to the junction between the 5′ end and the 3′ end of the polynucleotide as formed by and including the adapter polynucleotide.

The circularized polynucleotides are amplified, in some cases, for example, after degradation of the ligase enzyme, to yield amplified polynucleotides. Amplifying the circular polynucleotides in (b) can be effected by a polymerase. In some cases, the polymerase is a polymerase having strand-displacement activity. In some cases, the polymerase is a Phi29 DNA polymerase. Alternatively, the polymerase is a polymerase that does not have strand-displacement activity. In some cases, the polymerase is a T4 DNA polymerase or a T7 DNA polymerase. Alternately or in combination, the polymerase is a Taq polymerase, or polymerase in the Taq polymerase family. In some cases, amplification comprises rolling circle amplification (RCA). The amplified polynucleotides resulting from RCA can comprise linear concatemers, or polynucleotides comprising more than one copy of a target sequence (e.g., subunit sequence) from a template polynucleotide. In some embodiments, amplifying comprises subjecting the circular polynucleotides to an amplification reaction mixture comprising random primers. In some cases, amplifying comprises subjecting the circular polynucleotides to an amplification reaction mixture comprising one or more primers, each of which specifically hybridizes to a different target sequence via sequence complementarity. In some cases, amplifying comprises subjecting the circular polynucleotides to an amplification reaction mixture comprising inverse primers.

The amplified polynucleotides are sheared, in some cases, to produce sheared polynucleotides that are shorter in length relative to the unsheared polynucleotides. Two or more sheared polynucleotides originating from the same linear concatemer may have the same junction sequence but can have different 5′ and/or 3′ ends (e.g., shear ends).

Cell-free polynucleotides from a sample may be any of a variety of polynucleotides, including but not limited to, DNA, RNA, ribosomal RNA (rRNA), transfer RNA (tRNA), micro RNA (miRNA), messenger RNA (mRNA), fragments of any of these, or combinations of any two or more of these. In some embodiments, samples comprise DNA. In some embodiments, samples comprise cell-free genomic DNA. In some embodiments, the samples comprise DNA generated by amplification, such as by primer extension reactions using any suitable combination of primers and a DNA polymerase, including but not limited to polymerase chain reaction (PCR), reverse transcription, and combinations thereof Where the template for the primer extension reaction is RNA, the product of reverse transcription is referred to as complementary DNA (cDNA). Primers useful in primer extension reactions can comprise sequences specific to one or more targets, random sequences, partially random sequences, and combinations thereof. In general, sample polynucleotides comprise any polynucleotide present in a sample, which may or may not include target polynucleotides. The polynucleotides may be single-stranded, double-stranded, or a combination of these. In some embodiments, polynucleotides subjected to a method of the disclosure are single-stranded polynucleotides, which may or may not be in the presence of double-stranded polynucleotides. In some embodiments, the polynucleotides are single-stranded DNA. Single-stranded DNA (ssDNA) may be ssDNA that is isolated in a single-stranded form, or DNA that is isolated in double-stranded form and subsequently made single-stranded for the purpose of one or more steps in a method of the disclosure.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, such as cell-free DNA and cell-free RNA, which may correspond to dead or damaged cells. The identity of such cells may be used to characterize the cells or population of cells from which they are derived, such as tumor cells (e.g. in cancer detection), fetal cells (e.g. in prenatal diagnostic), cells from transplanted tissue (e.g. in early detection of transplant failure), or members of a microbial community.

If a sample is treated to extract polynucleotides, such as from cells in a sample, a variety of extraction methods are available. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993, which is entirely incorporated herein by reference), with or without the use of an automated nucleic acid extractor, e.g., the Model 341 DNA Extractor available from Applied Biosystems (Foster City, Calif); (2) stationary phase adsorption methods (U.S. Pat. No. 5,234,809; Walsh et al., 1991, each of which is entirely incorporated herein by reference); and (3) salt-induced nucleic acid precipitation methods (Miller et al., (1988) which is entirely incorporated herein by reference), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g. U.S. Pat. No. 5,705,628, which is entirely incorporated herein by reference). In some embodiments, the above isolation methods may be preceded by an enzyme digestion step to help eliminate unwanted protein from the sample, e.g., digestion with proteinase K, or other like proteases. See, e.g., U.S. Pat. No. 7,001,724, which is entirely incorporated herein by reference. If desired, RNase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic acid isolation step, purification of nucleic acids can be performed after any step in the disclosed methods, such as to remove excess or unwanted reagents, reactants, or products. A variety of methods for determining the amount and/or purity of nucleic acids in a sample are available, such as by absorbance (e.g. absorbance of light at 260 nm, 280 nm, and a ratio of these) and detection of a label (e.g. fluorescent dyes and intercalating agents, such as SYBR green, SYBR blue, DAPI, propidium iodine, Hoechst stain, SYBR gold, ethidium bromide).

In some cases, methods herein comprise preparation of a DNA library from polynucleotides. For example, methods herein comprise preparation of a single stranded DNA library. Any suitable method of preparing a single stranded DNA library may be used in methods herein. For example, the method of preparing a single stranded DNA library comprises denaturing the DNA sample to create a plurality of ssDNA; ligating an adapter to the 3′ end of the ssDNA molecules; synthesizing a second strand using a primer complementary to the adapter; ligating a double stranded adapter to the extension products; amplifying the second strand using primers targeting the first and second adapters (for example, using PCR); and sequencing the library on a sequencer. An additional method of single stranded library preparation comprises denaturing the DNA sample to create a plurality of ssDNA; ligating an adapter to the 3′ end of the ssDNA molecules; synthesizing the second strand by using a primer complementary to the adapter; ligating a double stranded adapter to the extension products; amplifying the second strand (for example, by PCR) the second strand using primers targeting the first and second adapters; optionally enriching for the regions of interest using hybridization with capture probes; amplifying (for example, by PCR) the captured products; and sequencing the library on a sequencer.

Further examples of single stranded library preparation include a method comprising the steps of treating the DNA with a heat labile phosphatase to remove residual phosphate groups from the 5′ and 3′ ends of the DNA strands; removal of deoxyuracils derived from cytosine deamination from the DNA strands; ligation of a 5′-phosphorylated adapter oligonucleotide having about 10 nucleotides and a long 3′ biotinylated spacer arm to the 3′ ends of the DNA strands; immobilization of adapter-ligated molecules on streptavidin beads; copying the template strand using a 5′-tailed primer complementary to the adapter using Bst polymerase; washing away excess primers; removal of 3′ overhangs using T4 DNA polymerase; joining a second adapter to the newly synthesized strands using blunt-end ligation; washing away excess adapter; releasing library molecules by heat denaturation; adding full-length adapter sequences including bar codes through amplification using tailed primers; and sequencing the library, as described in Gansauge et al. 2013. Nature Protocols. 8(4) 737-748, which is entirely incorporated herein by reference.

In additional embodiments, methods herein comprise preparation of a double stranded DNA library. Any suitable method of preparing a double stranded DNA library may be used in methods herein. For example, the method of preparing a double stranded DNA library comprises ligating sequencing adapters to the 5′ and 3′ ends of a plurality of DNA fragments and sequencing the library on a sequencer. An additional method of double stranded DNA library preparation comprises ligating adapters to the 5′ and 3′ ends of a plurality of DNA fragments; attaching the full adapter sequences to the ligated fragments through PCR using primers that are complementary to the ligated adapters; and sequencing the library on a sequencer. A further method comprises ligating adapters to the 5′ and 3′ ends of a plurality of DNA fragments; amplifying the ligated product through PCR that are complementary to the ligated adapters; optionally enriching for the regions of interest through hybridization with capture probes; PCR amplifying the captured products; and sequencing the library on a sequencer. An additional method of double stranded library preparation comprises ligating adapters to the 5′ and 3′ ends of a plurality of DNA fragments; amplifying the ligated product through PCR using primers that are complementary to the ligated adapters; circularizing the double stranded PCR products or denature and circularize the single stranded PCR products; optionally enriching for the regions of interest by PCR using primers targeting specific genes; and sequencing the library on a sequencer.

Further examples of double stranded library preparation include the Safe-Sequencing System described in Kinde et al. (Kinde et al. 2011. Proc. Natl. Acad. Sci., USA, 108(23) 9530-9535, which is entirely incorporated herein by reference) which comprises assignment of a unique identifier (UID) to each template molecule; amplification of each uniquely tagged template molecule to create UID families; and redundant sequencing of the amplification products. An additional example comprises the circulating single-molecule amplification and resequencing technology (cSMART) described in Lv et al. (Lv et al. 2015. Clin. Chem., 61(1) 172-181, which is entirely incorporated herein by reference) which tags single molecules with unique barcodes, circularizes, targets alleles for replication by inverse PCR, then sequencing the prepared library and counts the alleles present.

In additional library preparation methods, cfDNA fragments having certain features are selected using an antibody. In some cases, cfDNA fragments that are methylated or hypermethylated are selected using an antibody. Selected cfDNA fragments are then used in any library preparation method described herein, including circularization, single stranded DNA library preparation, and double stranded DNA library preparation. Sequencing such isolated cfDNA fragments provides information as to the features present in the cfDNA, including modifications such as methylation or hypermethylation.

According to some embodiments, polynucleotides among the plurality of polynucleotides from a sample are circularized. Circularization can include joining the 5′ end of a polynucleotide to the 3′ end of the same polynucleotide, to the 3′ end of another polynucleotide in the sample, or to the 3′ end of a polynucleotide from a different source (e.g. an artificial polynucleotide, such as an oligonucleotide adapter). In some embodiments, the 5′ end of a polynucleotide is joined to the 3′ end of the same polynucleotide (also referred to as “self-joining”). In some embodiment, conditions of the circularization reaction are selected to favor self-joining of polynucleotides within a particular range of lengths, so as to produce a population of circularized polynucleotides of a particular average length. For example, circularization reaction conditions may be selected to favor self-joining of polynucleotides shorter than about 5000, 2500, 1000, 750, 500, 400, 300, 200, 150, 100, 50, or fewer nucleotides in length. In some embodiments, fragments having lengths between 50-5000 nucleotides, 100-2500 nucleotides, or 150-500 nucleotides are favored, such that the average length of circularized polynucleotides falls within the respective range. In some embodiments, 80% or more of the circularized fragments are between 50-500 nucleotides in length, such as between 50-200 nucleotides in length. Reaction conditions that may be optimized include the length of time allotted for a joining reaction, the concentration of various reagents, and the concentration of polynucleotides to be joined. In some embodiments, a circularization reaction preserves the distribution of fragment lengths present in a sample prior to circularization. For example, one or more of the mean, median, mode, and standard deviation of fragment lengths in a sample before circularization and of circularized polynucleotides are within 75%, 80%, 85%, 90%, 95%, or more of one another.

In some cases, rather than preferentially forming self-joining circularization products, one or more adapter oligonucleotides are used, such that the 5′ end and 3′ end of a polynucleotide in the sample are joined by way of one or more intervening adapter oligonucleotides to form a circular polynucleotide. For example, the 5′ end of a polynucleotide can be joined to the 3′ end of an adapter, and the 5′ end of the same adapter can be joined to the 3′ end of the same polynucleotide. An adapter oligonucleotide includes any oligonucleotide having a sequence, at least a portion of which is known, that can be joined to a sample polynucleotide. Adapter oligonucleotides can comprise DNA, RNA, nucleotide analogues, non-canonical nucleotides, labeled nucleotides, modified nucleotides, or combinations thereof Adapter oligonucleotides can be single-stranded, double-stranded, or partial duplex. In general, a partial-duplex adapter comprises one or more single-stranded regions and one or more double-stranded regions. Double-stranded adapters can comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. When two hybridized regions of an adapter are separated from one another by a non-hybridized region, a “bubble” structure results. Adapters of different kinds can be used in combination, such as adapters of different sequences. Different adapters can be joined to sample polynucleotides in sequential reactions or simultaneously. In some embodiments, identical adapters are added to both ends of a target polynucleotide. For example, first and second adapters can be added to the same reaction. Adapters can be manipulated prior to combining with sample polynucleotides. For example, terminal phosphates can be added or removed.

Where adapter oligonucleotides are used, the adapter oligonucleotides can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as flow cells as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof. In some cases, the adapters may be used to purify those circles that contain the adapters, for example by using beads (particularly magnetic beads for ease of handling) that are coated with oligonucleotides comprising a complementary sequence to the adapter, that can “capture” the closed circles with the correct adapters by hybridization thereto, wash away those circles that do not contain the adapters and any unligated components, and then release the captured circles from the beads. In addition, in some cases, the complex of the hybridized capture probe and the target circle can be directly used to generate concatamers, such as by direct rolling circle amplification (RCA). In some embodiments, the adapters in the circles can also be used as a sequencing primer. Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the adapter oligonucleotide. A sequence element may be of any suitable length, such as about or less than about 3, 4, 5, 6, 7, 8, 9, 10, 15, 20, 25, 30, 35, 40, 45, 50 or more nucleotides in length. Adapter oligonucleotides can have any suitable length, at least sufficient to accommodate the one or more sequence elements of which they are comprised. In some embodiments, adapters are about or less than about 10, 15, 20, 25, 30, 35, 40, 45, 50, 55, 60, 65, 70, 75, 80, 90, 100, 200, or more nucleotides in length. In some embodiments, an adapter oligonucleotide is in the range of about 12 to 40 nucleotides in length, such as about 15 to 35 nucleotides in length.

In some embodiments, the adapter oligonucleotides joined to fragmented polynucleotides from one sample comprise one or more sequences common to all adapter oligonucleotides and a barcode that is unique to the adapters joined to polynucleotides of that particular sample, such that the barcode sequence can be used to distinguish polynucleotides originating from one sample or adapter joining reaction from polynucleotides originating from another sample or adapter joining reaction. In some embodiments, an adapter oligonucleotide comprises a 5′ overhang, a 3′ overhang, or both that is complementary to one or more target polynucleotide overhangs. Complementary overhangs can be one or more nucleotides in length, including but not limited to 1, 2, 3, 4, 5, 6, 7, 8, 9, 10, 11, 12, 13, 14, 15, or more nucleotides in length. Complementary overhangs may comprise a fixed sequence. Complementary overhangs of an adapter oligonucleotide may comprise a random sequence of one or more nucleotides, such that one or more nucleotides are selected at random from a set of two or more different nucleotides at one or more positions, with each of the different nucleotides selected at one or more positions represented in a pool of adapters with complementary overhangs comprising the random sequence. In some embodiments, an adapter overhang is complementary to a target polynucleotide overhang produced by restriction endonuclease digestion. In some embodiments, an adapter overhang consists of an adenine or a thymine.

A variety of methods for circularizing polynucleotides are available. In some embodiments, circularization comprises an enzymatic reaction, such as use of a ligase (e.g. an RNA or DNA ligase). A variety of ligases are available, including, but not limited to, Circligase™ (Epicentre; Madison, Wis.), RNA ligase, T4 RNA Ligase 1 (ssRNA Ligase, which works on both DNA and RNA). In addition, T4 DNA ligase can also ligate ssDNA if no dsDNA templates are present, although this is generally a slow reaction. Other non-limiting examples of ligases include NAD-dependent ligases including Taq DNA ligase, Thermus fihformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof Where self-joining is desired, the concentration of polynucleotides and enzyme can be adjusted to facilitate the formation of intramolecular circles rather than intermolecular structures. Reaction temperatures and times can be adjusted as well. In some embodiments, 60° C. is used to facilitate intramolecular circles. In some embodiments, reaction times are between 12-16 hours. Reaction conditions may be those specified by the manufacturer of the selected enzyme. In some embodiments, an exonuclease step can be included to digest any unligated nucleic acids after the circularization reaction. That is, closed circles do not contain a free 5′ or 3′ end, and thus the introduction of a 5′ or 3′ exonuclease will not digest the closed circles but will digest the unligated components. This may find particular use in multiplex systems.

In general, joining ends of a polynucleotide to one-another to form a circular polynucleotide (either directly, or with one or more intermediate adapter oligonucleotides) produces a junction having a junction sequence. Where the 5′ end and 3′ end of a polynucleotide are joined via an adapter polynucleotide, the term “junction” can refer to a junction between the polynucleotide and the adapter (e.g. one of the 5′ end junction or the 3′ end junction), or to the junction between the 5′ end and the 3′ end of the polynucleotide as formed by and including the adapter polynucleotide. Where the 5′ end and the 3′ end of a polynucleotide are joined without an intervening adapter (e.g. the 5′ end and 3′ end of a single-stranded DNA), the term “junction” refers to the point at which these two ends are joined. A junction may be identified by the sequence of nucleotides comprising the junction (also referred to as the “junction sequence”). In some embodiments, samples comprise polynucleotides having a mixture of ends formed by natural degradation processes (such as cell lysis, cell death, and other processes by which DNA is released from a cell to its surrounding environment in which it may be further degraded, such as in cell-free polynucleotides, such as cell-free DNA and cell-free RNA), fragmentation that is a byproduct of sample processing (such as fixing, staining, and/or storage procedures), and fragmentation by methods that cleave DNA without restriction to specific target sequences (e.g. mechanical fragmentation, such as by sonication; non-sequence specific nuclease treatment, such as DNase I, fragmentase). Where samples comprise polynucleotides having a mixture of ends, the likelihood that two polynucleotides will have the same 5′ end or 3′ end is low, and the likelihood that two polynucleotides will independently have both the same 5′ end and 3′ end is extremely low. Accordingly, in some embodiments, junctions may be used to distinguish different polynucleotides, even where the two polynucleotides comprise a portion having the same target sequence. Where polynucleotide ends are joined without an intervening adapter, a junction sequence may be identified by alignment to a reference sequence. For example, where the order of two component sequences appears to be reversed with respect to the reference sequence, the point at which the reversal appears to occur may be an indication of a junction at that point. Where polynucleotide ends are joined via one or more adapter sequences, a junction may be identified by proximity to the known adapter sequence, or by alignment as above if a sequencing read is of sufficient length to obtain sequence from both the 5′ and 3′ ends of the circularized polynucleotide. In some embodiments, the formation of a particular junction is a sufficiently rare event such that it is unique among the circularized polynucleotides of a sample.

### Methods of Sequencing

According to some embodiments, linear and/or circularized polynucleotides (or amplification products thereof, which may have optionally been enriched) are subjected to a sequencing reaction to generate sequencing reads. Sequencing reads produced by such methods may be used in accordance with other methods disclosed herein. A variety of sequencing methodologies are available, particularly high-throughput sequencing methodologies. Examples include, without limitation, sequencing systems manufactured by Illumina (sequencing systems such as HiSeq® and MiSeq®), Life Technologies (Ion Torrent®, SOLiD®, etc.), Roche's 454 Life Sciences systems, Pacific Biosciences systems, Oxford Nanopore Technologies, nanoball sequencing, sequencing by hybridization, polymerized colony (POLONY) sequencing, nanogrid rolling circle sequencing (ROLONY), etc. In some embodiments, sequencing comprises use of HiSeq® and MiSeq® systems to produce reads of about or more than about 50, 75, 100, 125, 150, 175, 200, 250, 300, or more nucleotides in length. In some embodiments, sequencing comprises a sequencing by synthesis process, where individual nucleotides are identified iteratively, as they are added to the growing primer extension product. Pyrosequencing is an example of a sequence by synthesis process that identifies the incorporation of a nucleotide by assaying the resulting synthesis mixture for the presence of by-products of the sequencing reaction, namely pyrophosphate. In particular, a primer/template/polymerase complex is contacted with a single type of nucleotide. If that nucleotide is incorporated, the polymerization reaction cleaves the nucleoside triphosphate between the a and 13 phosphates of the triphosphate chain, releasing pyrophosphate. The presence of released pyrophosphate is then identified using a chemiluminescent enzyme reporter system that converts the pyrophosphate, with AMP, into ATP, then measures ATP using a luciferase enzyme to produce measurable light signals. Where light is detected, the base is incorporated, where no light is detected, the base is not incorporated. Following appropriate washing steps, the various bases are cyclically contacted with the complex to sequentially identify subsequent bases in the template sequence. See, e.g., U.S. Pat. No. 6,210,891.

In related sequencing processes, the primer/template/polymerase complex is immobilized upon a substrate and the complex is contacted with labeled nucleotides. The immobilization of the complex may be through the primer sequence, the template sequence and/or the polymerase enzyme, and may be covalent or noncovalent. For example, immobilization of the complex can be via a linkage between the polymerase or the primer and the substrate surface. In alternate configurations, the nucleotides are provided with and without removable terminator groups. Upon incorporation, the label is coupled with the complex and is thus detectable. In the case of terminator bearing nucleotides, all four different nucleotides, bearing individually identifiable labels, are contacted with the complex. Incorporation of the labeled nucleotide arrests extension, by virtue of the presence of the terminator, and adds the label to the complex, allowing identification of the incorporated nucleotide. The label and terminator are then removed from the incorporated nucleotide, and following appropriate washing steps, the process is repeated. In the case of non-terminated nucleotides, a single type of labeled nucleotide is added to the complex to determine whether it will be incorporated, as with pyrosequencing. Following removal of the label group on the nucleotide and appropriate washing steps, the various different nucleotides are cycled through the reaction mixture in the same process. See, e.g., U.S. Pat. No. 6,833,246, incorporated herein by reference in its entirety for all purposes. For example, the Illumina Genome Analyzer System is based on technology described in WO 98/44151, wherein DNA molecules are bound to a sequencing platform (flow cell) via an anchor probe binding site (otherwise referred to as a flow cell binding site) and amplified in situ on a glass slide. A solid surface on which DNA molecules are amplified typically comprise a plurality of first and second bound oligonucleotides, the first complementary to a sequence near or at one end of a target polynucleotide and the second complementary to a sequence near or at the other end of a target polynucleotide. This arrangement permits bridge amplification, such as described in US20140121116. The DNA molecules are then annealed to a sequencing primer and sequenced in parallel base-by-base using a reversible terminator approach. Hybridization of a sequencing primer may be preceded by cleavage of one strand of a double-stranded bridge polynucleotide at a cleavage site in one of the bound oligonucleotides anchoring the bridge, thus leaving one single strand not bound to the solid substrate that may be removed by denaturing, and the other strand bound and available for hybridization to a sequencing primer. Typically, the Illumina Genome Analyzer System utilizes flow-cells with 8 channels, generating sequencing reads of 18 to 36 bases in length, generating >1.3 Gbp of high quality data per run (see www.illumina.com).

In yet a further sequence by synthesis process, the incorporation of differently labeled nucleotides is observed in real time as template dependent synthesis is carried out. An individual immobilized primer/template/polymerase complex may be observed as fluorescently labeled nucleotides are incorporated, permitting real time identification of each added base as it is added. In this process, label groups may be attached to a portion of the nucleotide that is cleaved during incorporation. For example, by attaching the label group to a portion of the phosphate chain removed during incorporation, i.e., a β,γ, or other terminal phosphate group on a nucleoside polyphosphate, the label is not incorporated into the nascent strand, and instead, natural DNA is produced. Observation of individual molecules may involve the optical confinement of the complex within a very small illumination volume. By optically confining the complex, a monitored region may be created, in which randomly diffusing nucleotides may be present for a very short period of time, while incorporated nucleotides may be retained within the observation volume for longer as they are being incorporated. This may result in a characteristic signal associated with the incorporation event, which is also characterized by a signal profile that is characteristic of the base being added. Interacting label components, such as fluorescent resonant energy transfer (FRET) dye pairs, may be provided with the polymerase or other portion of the complex and the incorporating nucleotide, such that the incorporation event puts the labeling components in interactive proximity, and a characteristic signal results, that is again, also characteristic of the base being incorporated (See, e.g., U.S. Pat. Nos. 6,917,726, 7,033,764, 7,052,847, 7,056,676, 7,170,050, 7,361,466, and 7,416,844; and US 20070134128, each of which is entirely incorporated herein by reference).

In some embodiments, the nucleic acids in the sample can be sequenced by ligation. This method typically uses a DNA ligase enzyme to identify the target sequence, for example, as used in the polony method and in the SOLiD technology (Applied Biosystems, now Invitrogen). In general, a pool of all possible oligonucleotides of a fixed length is provided, labeled according to the sequenced position. Oligonucleotides are annealed and ligated; the preferential ligation by DNA ligase for matching sequences results in a signal corresponding to the complementary sequence at that position.

Sequencing methods of the present disclosure may provide information useful for various applications, such as, for example, identifying a disease (e.g., cancer) in a subject or determining that the subject is at risk of having (or developing) the disease. Sequencing may provide a sequence of a polymorphic region. Sequencing may provide a length of a polynucleotide, such as a DNA (e.g., cfDNA). Further, sequencing may provide a sequence of a breakpoint or end of a DNA, such as a cfDNA. Sequencing may provide a sequence of a border of a protein binding site or a border of a DNase hypersensitive site.

### Samples

In some embodiments of the various methods described herein, the sample is from a subject. A subject may be any animal, including but not limited to, a cow, a pig, a mouse, a rat, a chicken, a cat, a dog, etc., and is usually a mammal, such as a human. Sample polynucleotides are often isolated from a cell-free sample from a subject, such as a tissue sample, bodily fluid sample, or organ sample, including, for example, blood sample, or fluid sample containing nucleic acids (e.g. saliva). In some cases, the sample is treated to remove cells, or polynucleotides are isolated without a cellular extractions step (e.g. to isolate cell-free polynucleotides, such as cell-free DNA). Other examples of sample sources include those from blood, urine, feces, nares, the lungs, the gut, other bodily fluids or excretions, materials derived therefrom, or combinations thereof. In some embodiments, the sample is a blood sample or a portion thereof (e.g. blood plasma or serum). Serum and plasma may be of particular interest, due to the relative enrichment for tumor DNA associated with the higher rate of malignant cell death among such tissues. In some embodiments, a sample from a single individual is divided into multiple separate samples (e.g. 2, 3, 4, 5, 6, 7, 8, 9, 10, or more separate samples) that are subjected to methods of the disclosure independently, such as analysis in duplicate, triplicate, quadruplicate, or more. Where a sample is from a subject, the reference sequence may also be derived from the subject, such as a consensus sequence from the sample under analysis or the sequence of polynucleotides from another sample or tissue of the same subject. For example, a blood sample may be analyzed for cfDNA mutations, while cellular DNA from another sample (e.g. buccal or skin sample) is analyzed to determine the reference sequence.

Polynucleotides may be extracted from a sample according to any suitable method. A variety of kits are available for extraction of polynucleotides, selection of which may depend on the type of sample, or the type of nucleic acid to be isolated. Examples of extraction methods are provided herein, such as those described with respect to any of the various aspects disclosed herein. In one example, the sample may be a blood sample, such as a sample collected in an EDTA tube (e.g. BD Vacutainer). Plasma can be separated from the peripheral blood cells by centrifugation (e.g. 10 minutes at 1900×g at 4° C.). Plasma separation performed in this way on a 6mL blood sample will typically yield 2.5 to 3 mL of plasma. Circulating cell-free DNA can be extracted from a plasma sample, such as by using a QIAmp Circulating Nucleic Acid Kit (Qiagene), according the manufacturer's protocol. DNA may then be quantified (e.g. on an Agilent 2100 Bioanalyzer with High Sensitivity DNA kit (Agilent)). As an example, yield of circulating DNA from such a plasma sample from a healthy person may range from ing to lOng per mL of plasma, with significantly more in disease (e.g., cancer) patient samples.

In some embodiments, the plurality of polynucleotides comprises cell-free polynucleotides, such as cell-free DNA (cfDNA), cell-free RNA (cfRNA), circulating tumor DNA (ctDNA), or circulating tumor RNA (ctRNA). Cell-free DNA circulates in both healthy and diseased individuals. Cell-free RNA circulates in both healthy and diseased individuals. cfDNA from tumors (ctDNA) is not confined to any specific cancer type, but appears to be a common finding across different malignancies. According to some measurements, the free circulating DNA concentration in plasma is about 14-18 ng/ml in control subjects and about 180-318 ng/ml in patients with neoplasia. Apoptotic and necrotic cell death contribute to cell-free circulating DNA in bodily fluids. For example, significantly increased circulating DNA levels have been observed in plasma of prostate cancer patients and other prostate diseases, such as Benign Prostate Hyperplasia and Prostatits. In addition, circulating tumor DNA is present in fluids originating from the organs where the primary tumor occurs. Thus, breast cancer detection can be achieved in ductal lavages; colorectal cancer detection in stool; lung cancer detection in sputum, and prostate cancer detection in urine or ejaculate. Cell-free DNA may be obtained from a variety of sources. One common source is blood samples of a subject. However, cfDNA or other fragmented DNA may be derived from a variety of other sources. For example, urine and stool samples can be a source of cfDNA, including ctDNA. Cell-free RNA may be obtained from a variety of sources.

In some embodiments, polynucleotides are subjected to subsequent steps (e.g. circularization and amplification) without an extraction step, and/or without a purification step. For example, a fluid sample may be treated to remove cells without an extraction step to produce a purified liquid sample and a cell sample, followed by isolation of DNA from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides. For example, cell-free polynucleotides may include cell-free DNA (also called “circulating” DNA). In some embodiments, the circulating DNA is circulating tumor DNA (ctDNA) from tumor cells, such as from a body fluid or excretion (e.g. blood sample). Cell-free polynucleotides may include cell-free RNA (also called “circulating” RNA). In some embodiments, the circulating RNA is circulating tumor RNA (ctRNA) from tumor cells. Tumors may show apoptosis or necrosis, such that tumor nucleic acids are released into the body, including the blood stream of a subject, through a variety of mechanisms, in different forms and at different levels. Typically, the size of the ctDNA can range between higher concentrations of smaller fragments, generally 70 to 200 nucleotides in length, to lower concentrations of large fragments of up to thousands kilobases.

### Cancer

Methods herein provide for detection of cancer or detection risk of cancer. Staging of cancer is dependent on cancer type where each cancer type has its own classification system. Examples of cancer staging or classification systems are described in more detail below.

TABLE 1
Colon Cancer Primary Tumor (T)

| | |
|---|---|
| TX | Primary tumor cannot be assessed |
| T0 | No evidence of primary tumor |
| Tis | Carcinoma in situ: intraepithelial or intramucosal carcinoma (involvement of lamina propria with |
| | no extension through the muscularis mucosa) |
| T1 | Tumor invades submucosa (through the muscularis mucosa but not into the muscularis propria) |
| T2 | Tumor invades muscularis propria |
| T3 | Tumor invades through the muscularis propria into the pericolorectal tissues |
| T4 | Tumor invades the visceral peritoneum or invades or adheres to adjacent organ or structure |
| T4a | Tumor invades through the visceral peritoneum (including gross perforation of the bowel |
| | through tumor and continuous invasion of tumor through areas of inflammation to the surface of |
| | the visceral peritoneum) |
| T4b | Tumor directly invades or is adherent to other organs or structures |

Colon Cancer Regional Lymph Notes (N)

| | |
|---|---|
| NX | Regional lymph nodes cannot be assessed |
| N0 | No regional lymph node metastasis |
| N1 | Metastasis in 1-3 regional lymph nodes (tumor in lymph nodes measuring ≥0.2 mm) or any |
| | number of tumor deposits are present and all identifiable nodes are negative |
| N1a | Metastasis in 1 regional lymph node |
| N1b | Metastasis in 2-3 regional lymph nodes |
| N1c | Tumor deposit(s) in the subserosa, mesentery, or nonperitonealized, pericolic, or perirectal/ |
| | mesorectal tissues without regional nodal metastasis |
| N2 | Metastasis in 4 or more lymph nodes |
| N2a | Metastasis in 4-6 regional lymph nodes |
| N2b | Metastasis in 7 or more regional lymph nodes |

Colon Cancer Distant Metastasis (M)

| | |
|---|---|
| M0 | No distant metastasis by imaging or other studies, no evidence of tumor in distant sites or organs. |
| | (This category is not assigned by pathologists.) |
| M1 | Metastasis to one or more distant sites or organs or peritoneal metastasis |
| M1a | Metastasis confined to 1 organ or site (e.g., liver, lung, ovary, nonregional node) without |
| | peritoneal metastasis |
| M1b | Metastasis to two or more sites or organs without peritoneal metastasis |
| M1c | Metastasis to the peritoneal surface alone or with other site or organ metastases |

TABLE 2
Colon Cancer Anatomic stage/prognostic groups

| Stage | T | N | M | Dukes | MAC |
|---|---|---|---|---|---|
| 0 | Tis | N0 | M0 | — | — |
| I | T1 | N0 | M0 | A | A |
| | T2 | N0 | M0 | A | B1 |
| IIA | T3 | N0 | M0 | B | B2 |
| IIB | T4a | N0 | M0 | B | B2 |
| IIC | T4b | N0 | M0 | B | B3 |
| IIIA | T1-T2 | N1/N1c | M0 | C | C1 |
| | T1 | N2a | M0 | C | C1 |
| IIIB | T3-T4a | N1/N1c | M0 | C | C2 |
| | T2-T3 | N2a | M0 | C | C1/C2 |
| | T1-T2 | N2b | M0 | C | C1 |
| IIIC | T4a | N2a | M0 | C | C2 |
| | T3-T4a | N2b | M0 | C | C2 |
| | T4b | N1-N2 | M0 | C | C3 |
| IVA | Any T | Any N | M1a | — | — |
| IVB | Any T | Any N | M1b | — | — |
| IVC | Any T | Any N | M1c | — | — |

TABLE 3
Malignant Melanoma Primary Tumor (T)

| | |
|---|---|
| TX | Primary tumor cannot be assessed (i.e. curettaged melanoma) |
| T0 | No evidence of primary tumor |
| Tis | Melanoma in situ |
| T1 | Thickness ≤1.0 mm |
| | T1a: <0.8 mm without ulceration |
| | T1b: <0.8 mm with ulceration, or 0.8-1.0 mm with or without ulceration |
| T2 | Thickness >1.0-2.0 mm |
| | T2a: Without ulceration |
| | T2b: With ulceration |
| T3 | Thickness >2.0-4.0 mm |
| | T3a: Without ulceration |
| | T3b: With ulceration |
| T4 | Thickness >4.0 mm |
| | T4a: Without ulceration |
| | T4b: With ulceration |

Malignant Melanoma Regional Lymph Notes (N)

| | |
|---|---|
| NX | Regional lymph nodes cannot be assessed |
| N0 | No regional metastasis detected |
| N1 | One tumor-involved lymph node or in-transit, satellite, and/or microsatellite metastases with no |
| | tumor-involved nodes |
| | N1a: One clinically occult (i.e., detected by sentinel lymph node biopsy [SLNB]; no in-transit, |
| | satellite, or microsatellite metastases |
| | N1b: One clinically detected; no in-transit, satellite, or microsatellite metastases |
| | N1c: No regional lymph node disease; in-transit, satellite, and/or microsatellite metastases found |
| N2 | Two or three tumor-involved nodes; or in-transit, satellite, or microsatellite metastases |
| | N2a: Two or three clinically occult (i.e., detected by SLNB); no in-transit, satellite, or |
| | microsatellite metastases |
| | N2b: Two or three clinically detected; no in-transit, satellite, or microsatellite metastases |
| | N2c: One clinically occult or clinically detected; in-transit, satellite, and/or microsatellite |
| | metastases found |
| N3 | ≥4 tumor-involved nodes or in -transit, satellite, and/or microsatellite metastases with ≥2 tumor- |
| | involved nodes or any number of matted nodes without or with in-transit, satellite, and/or |
| | microsatellite metastases |
| | N3a: ≥4 clinically occult (i.e., detected by SLNB); no in-transit, satellite, or microsatellite |
| | metastases |
| | N3b: ≥4, at least one of which was clinically detected, or presence of any matted nodes; no in- |
| | transit, satellite, or microsatellite metastases |
| | N3c: ≥2 clinically occult or clinically detected and/or presence of any matted nodes, with |
| | presence of in-transit, satellite, and/or microsatellite metastases |

Malignant Melanoma Distant Metastasis (M)

| | |
|---|---|
| M0 | No detectable evidence of distant metastases |
| M1a | Metastases to skin, soft tissue (including muscle), and/or nonregional lymph nodes |
| M1b | Lung metastasis, with or without M1a involvement |
| M1c | Distant metastasis to non-central nervous system (CNS) visceral sites with or without M1a or |
| | M1b involvement |
| M1d | Distant metastasis to CNS, with or without M1a or M1b involvement |

TABLE 4
Malignant Melanoma Anatomic stage/prognostic groups

| Stage | T | N | M |
|---|---|---|---|
| 0 | Tis | N0 | M0 |
| IA | T1a | N0 | M0 |
| IB | T1b | N0 | M0 |
| | T2a | N0 | M0 |
| IIA | T2b | N0 | M0 |
| | T3a | N0 | M0 |
| IIB | T3b | N0 | M0 |
| | T4a | N0 | M0 |
| IIC | T4b | N0 | M0 |
| III | Any T, Tis | N1, N2, or N3 | M0 |
| IV | Any T | Any N | M1 |

TABLE 5
Hepatocellular Carcinoma Primary tumor (T)

| | |
|---|---|
| TX | Primary tumor cannot be assessed |
| T0 | No evidence of primary tumor |
| T1 | Solitary tumor 2 cm without vascular invasion |
| T1a | Solitary tumor <2 cm |
| T1b | Solitary tumor >2 cm without vascular invasion |
| T2 | Solitary tumor >2 cm with vascular invasion; or multiple tumors, non >5 cm |
| T3 | Multiple tumors, at least one of which is >5 cm |
| T4 | Single tumor or tumors of any size involving a major branch of the portal vein or |
| | hepatic vein, or tumor(s) with direct invasion of adjacent organs other than the |
| | gallbladder or with perforation of visceral peritoneum |

Hepatocellular Carcinoma Regional Lymph Nodes (N)

| | |
|---|---|
| NX | Regional lymph node(s) cannot be assessed |
| N0 | No regional lymph node metastasis |
| N1 | Regional lymph node metastasis |

Hepatocellular Carcinoma Distant Metastasis (M)

| | |
|---|---|
| M0 | No distant metastasis |
| M1 | Distant metastasis |

TABLE 6
Hepatocellular Carcinoma Anatomic stage/prognostic groups

| Stage | T | N | M |
|---|---|---|---|
| IA | T1a | N0 | M0 |
| IB | T1b | N0 | M0 |
| II | T2 | N0 | M0 |
| IIIA | T3 | N0 | M0 |
| IIIB | T4 | N0 | M0 |
| IVA | Any T | N1 | M0 |
| IVB | Any T | Any N | M1 |

TABLE 7
Hepatocellular Carcinoma Histologic grade

| | |
|---|---|
| GX | Grade cannot be accessed |
| G1 | Well differentiated |
| G2 | Moderately differentiated |
| G3 | Poorly differentiated |
| G4 | Undifferentiated |

TABLE 8
Barcelona-Clinic Liver Cancer staging system

| | Performance | | Okuda | |
|---|---|---|---|---|
| Stage | Status | Tumor Stage | Stage | Liver function |
| A: Early HCC | | | | |
| A1 | 0 | Single, <5 cm | I | No portal |
| | | | | hypertension, normal |
| | | | | bilirubin |
| A2 | 0 | Single, <5 cm | I | Portal hypertension, |
| | | | | normal bilirubin |
| A3 | 0 | Single, <5 cm | I | Portal hypertension, |
| | | | | normal bilirubin |
| A4 | 0 | 3 tumors, <3 cm | I-II | Child-Pugh A-B |
| Stage B: Intermediate | 0 | Large, | I-II | Child-Pugh A-B |
| HCC | | multinodular | | |
| Stage C: Advanced | 1-2 | Vascular invasion | I-II | Child-Pugh A-B |
| HCC | | or extrahepatic | | |
| | | spread | | |
| Stage D: End-Stage | 3-4 | Any | I-II | Child-Pugh C |
| HCC | | | | |

TABLE 9
Ishak Fibrosis score

| Architectural Change | Score |
|---|---|
| No fibrosis | 0 |
| Fibrous expansion of some portal areas, with or | 1 |
| without short fibrous septa | |
| Fibrous expansion of most portal areas, with or | 2 |
| without short fibrous septa | |
| Fibrous expansion of portal areas with occasional | 3 |
| portal-to-portal bridging | |
| Fibrous expansion of portal areas with marked | 4 |
| bridging as well as portal-central | |
| Marked bridging (portal-to-portal and/or portal- | 5 |
| central) with occasional nodule (incomplete | |
| cirrhosis) | |
| Cirrhosis, probable or definite | 6 |

TABLE 10
Gastric Cancer Primary tumor (T)

| | |
|---|---|
| TX | Primary tumor cannot be assessed |
| T0 | No evidence of primary tumor |
| Tis | Carcinoma in situ: intraepithelial tumor without invasion of the lamina propria |
| T1 | Tumor invades lamina propria, muscularis mucosae, or submucosa |
| T1a | Tumor invades lamina propria or muscularis mucosae |
| T1b | Tumor invades submucosa |
| T2 | Tumor invades muscularis propria |
| T3 | Tumor penetrates subserosal connective tissue without invasion of visceral peritoneum |
| | or adjacent structures. |
| T4 | Tumor invades serosa (visceral peritoneum) or adjacent structures |
| T4a | Tumor invades serosa (visceral peritoneum) |
| T4b | Tumor invades adjacent structures |

Regional Lymph Nodes (N)

| | |
|---|---|
| NX | Regional lymph node(s) cannot be assessed |
| N0 | No regional lymph node metastasis |
| N1 | Metastasis in 1-2 regional lymph nodes |
| N2 | Metastasis in 3-6 regional lymph nodes |
| N3 | Metastasis in seven or more regional lymph nodes |
| N3a | Metastasis in 7-15 regional lymph nodes |
| N3b | Metastasis in 16 or more regional lymph nodes |

Distant Metastasis (M)

| | |
|---|---|
| M0 | No distant metastasis |
| M1 | Distant metastasis |

TABLE 11
Gastric Cancer Clinical stage/prognostic groups (cTNM)

| Stage | T | N | M |
|---|---|---|---|
| 0 | Tis | N0 | M0 |
| I | T1 | N0 | M0 |
| | T2 | N0 | M0 |
| IIA | T1 | N1, N2, N3 | M0 |
| | T2 | N1, N2, N3 | M0 |
| IIB | T3 | N0 | M0 |
| | T4 | N0 | M0 |
| III | T | N1, N2, N3 | M0 |
| | T4a | N1, N2, N3 | M0 |
| IVA | Any T | Any N | M0 |
| IVB | Any T | Any N | M1 |

TABLE 12
Gastric Cancer Pathological stage (pTNM)

| Stage | T | N | M |
|---|---|---|---|
| 0 | Tis | N0 | M0 |
| I | T1 | N0 | M0 |
| | T1 | N1 | M0 |
| IB | T2 | N0 | M0 |
| | T1 | N2 | M0 |
| II A | T2 | N1 | M0 |
| | T3 | N0 | M0 |
| | T1 | N3 | M0 |
| | T2 | N2 | M0 |

TABLE 13
Gastric Cancer Post-neoadjuvant therapy
staging and overall survival (ypTNM)

| | | | | 3-year | 5-year |
|---|---|---|---|---|---|
| Stage | T | N | M | survival (%) | survival (%) |
| I | T1, T2 | N0 | M0 | 81.4 | 76.5 |
| | T1 | N1 | M0 | | |
| | T1 | N2, N3 | M0 | | |
| | T2 | N1, N2 | M0 | | |
| II | T3 | N0, N1 | M0 | 54.8 | 46.3 |
| | T4a | N0 | M0 | | |
| | T2 | N3 | M0 | | |
| | T3 | M2, N3 | M0 | | |
| III | T4a | N1, N2, N3 | M0 | | |
| | T4b | N0, N1, N2, N3 | M0 | 28.8 | 18.3 |
| IV | Any T | Any N | M1 | 10.2 | 5.7 |

TABLE 14
Esophageal Cancer Primary tumor (T)

| | |
|---|---|
| TX | Primary tumor cannot be assessed |
| T0 | No evidence of primary tumor |
| Tis | High-grade dysplasia,* defined as malignant cells confined by |
| | the basement membrane |
| T1 | Tumor invades lamina propria, muscularis mucosae, or submucosa |
| T1a | Tumor invades lamina propria or muscularis mucosae |
| T1b | Tumor invades submucosa |
| T2 | Tumor invades muscularis propria |
| T3 | Tumor invades adventitia |
| T4 | Tumor invades adjacent structures |
| T4a | Resectable tumor invading pleura, pericardium, azygos vein, |
| | diaphragm or peritoneum |
| T4b | Unresectable tumor invading other adjacent structures, such as |
| | the aorta, vertebral body, and trachea |

Esophageal Cancer Regional Lymph Nodes (N)

| | |
|---|---|
| NX | Regional lymph node(s) cannot be assessed |
| N0 | No regional lymph node metastasis |
| N1 | Metastasis in 1-2 regional lymph nodes |
| N2 | Metastasis in 3-6 regional lymph nodes |
| N3 | Metastasis in 7 or more regional lymph nodes |

Esophageal Cancer Distant Metastasis (M)

| | |
|---|---|
| M0 | No distant metastasis |
| M1 | Distant metastasis |

TABLE 15
Esophageal Cancer Histologic grade
Histologic grade (G)

| | |
|---|---|
| GX | Grade cannot be assessed - stage grouping as G1 |
| G1 | Well differentiated |
| G2 | Moderately differentiated |
| G3 | Poorly differentiated or undifferentiated* |

TABLE 16
Squamous cell carcinoma location

| | |
|---|---|
| X | Location unknown |
| Upper | Cervical esophagus to lower border of azygos vein |
| Middle | Lower border of azygos vein to lower border of |
| | inferior pulmonary vein |
| Lower | Lower border of inferior pulmonary vein to stomach, |
| | including gastroesophageal junction |

TABLE 17
Esophageal Cancer Clinical stage groups

| | | | |
|---|---|---|---|
| Stage Group | cT | cN | cM |

Squamous cell carcinoma

| | | | |
|---|---|---|---|
| 0 | Tis | N0 | M0 |
| I | T1 | N0-1 | M0 |
| | T2 | N0-1 | M0 |
| II | T3 | N0 | M0 |
| | T3 | N1 | M0 |
| III | T1-3 | N2 | M0 |
| | T4 | N0-2 | M0 |
| IVA | T1-4 | N3 | M0 |
| IVB | T1-4 | N0-3 | M1 |

Adenocarcinoma

| | | | |
|---|---|---|---|
| 0 | Tis | N0 | M0 |
| I | T1 | N0 | M0 |
| IIA | T1 | N1 | M0 |
| IIB | T2 | N0 | M0 |
| | T2 | N1 | M0 |
| III | T3-4a | N0-1 | M0 |
| | T1-4a | N2 | M0 |
| IVA | T4b | N0-2 | M0 |
| | T1-4 | N3 | M0 |
| IVB | T1-4 | N0-3 | M1 |

TABLE 18
Pathologic stage groups (Open Table in a new window)

| | | | | | |
|---|---|---|---|---|---|
| Stage Group | pT | pN | pM | Grade | Location |

Squamous cell carcinoma

| | | | | | |
|---|---|---|---|---|---|
| 0 | Tis | N0 | M0 | N/A | Any |
| IA | T1a | N0 | M0 | G-1, X | Any |
| | T1b | N0 | M0 | G1-3, X | Any |
| IB | T1a | N0 | M0 | G2-3 | Any |
| | T2 | N0 | M0 | G1 | Any |
| | T2 | N0 | M0 | G2-3, X | Any |
| IIA | T3 | N0 | M0 | Any | Lower |
| | T34 | N0 | M0 | G1 | Upper/middle |
| | T3 | N0 | M0 | G2-3 | Upper/middle |
| | T3 | N0 | M0 | GX | Any |
| IIB | T3 | N0 | M0 | Any | X |
| | T1 | N1 | M0 | Any | Any |
| IIIA | T1 | N2 | M0 | Any | Any |
| | T2 | N1 | M0 | Any | Any |
| | T4a | N0-1 | M0 | Any | Any |
| IIIB | T3 | N1 | M0 | Any | Any |
| | T2-3 | N2 | M0 | Any | Any |
| | T4a | N2 | M0 | Any | Any |
| IVA | T4b | N0-2 | M0 | Any | Any |
| | T1-4 | N3 | M0 | Any | Any |
| IVB | T1-4 | N0-3 | M1 | Any | Any |

Adenocarcinoma

| | | | | |
|---|---|---|---|---|
| 0 | Tis | N0 | M0 | N/A |
| IA | T1a | N0 | M0 | G1, X |
| IB | T1a | N0 | M0 | G2 |
| | T1b | N0 | M0 | G1-2, X |
| | T1 | N0 | M0 | G3 |
| IC | T2 | N0 | M0 | G1-2 |
| IIA | T2 | N0 | M0 | G3, X |
| | T1 | N1 | M0 | Any |
| IIB | T3 | N0 | M0 | Any |
| | T1 | N2 | M0 | Any |
| IIIA | T2 | N1 | M0 | Any |
| | T4a | N0-1 | M0 | Any |
| IIIB | T3 | N1 | M0 | Any |
| | T2-3 | N2 | M0 | Any |
| IVA | T4a | N2 | M0 | Any |
| | T4b | N0-2 | M0 | Any |
| | T1-4 | N3 | M0 | Any |
| | R1-4 | N0-3 | M1 | Any |

TABLE 19
Postneoadjuvant therapy staging (Open Table in a new window)

| | | | |
|---|---|---|---|
| Stage Group | ypT | ypN | ypM |

Squamous cell carcinoma

| | | | |
|---|---|---|---|
| I | T0-2 | N0 | M0 |
| II | T3 | N0 | M0 |
| IIIA | T0-2 | N1 | M0 |
| | T4a | N0 | M0 |
| IIIB | T3 | N1 | M0 |
| | T0-3 | N2 | M0 |
| | T4a | N1-2, X | M0 |
| IVA | T4b | N0-2 | M0 |
| | T1-4 | N3 | M0 |
| IVB | T1-4 | N0-3 | M1 |

TABLE 20

| | | |
|---|---|---|
| TNM | FIGO stages | Surgical-pathologic findings |

Endometrial Cancer Primary Tumor (T)

| | | |
|---|---|---|
| TX | | Primary tumor cannot be assessed |
| T0 | | No evidence of primary tumor |
| Tis | | Carcinoma in situ (preinvasive carcinoma) |
| T1 | I | Tumor confined to corpus uteri |
| T1a | IA | Tumor linked to endometrium or invades less than one half of |
| | | the myometrium |
| T1b | IB | Tumor invades one half or more of the myometrium |
| T2 | II | Tumor invades stromal connective tissue of the cervix but does |
| | | not extend beyond uterus** |
| T3a | IIIA | Tumor involves serosa and/or adnexa (direct extension or |
| | | metastasis) |
| T3b | IIIB | Vaginal involvement (direct extension or metastasis) or |
| | | parametrial involvement |
| | IIIC | Metastases to pelvic and/or para-aortic lymph nodes |
| | IV | Tumor invades bladder mucosa and/or bowel mucosa, and/or |
| | | distant metastases |
| T4 | IVA | Tumor invades bladder mucosa and/or bowel mucosa (bullous |
| | | edema is not sufficient to classify a tumor as T4) |

Endometrial Cancer Regional Lymph Nodes (N)

| | | |
|---|---|---|
| TNM | FIGO | Surgical-pathologic findings |
| | stages | |
| NX | | Regional lymph nodes cannot be assessed |
| N0 | | No regional lymph node metastasis |
| N1 | IIIC1 | Regional lymph node metastasis to pelvic lymph nodes |
| N2 | IIIC2 | Regional lymph node metastasis to para-aortic lymph nodes, |
| | | with or without positive pelvic lymph nodes |

Endometrial Cancer Distant Metastasis

| | | |
|---|---|---|
| TNM | FIGO | Surgical-pathologic findings |
| | stages | |
| M0 | | No distant metastasis |
| M1 | | Distant metastasis (includes metastasis to inguinal lymph nodes, |
| | | intraperitoneal M1 IVB disease, or lung, liver, or bone |
| | | metastases; it excludes metastasis to para-aortic lymph nodes, |
| | | vagina, pelvic serosa, or adnexa) |

TABLE 21
Non-Small Cell Lung Cancer Primary tumor (T)

| | |
|---|---|
| TX | Primary tumor cannot be assessed, or tumor is proven by the presence of malignant cells in |
| | sputum or bronchial washings but not visualized by imaging or bronchoscopy |
| T0 | No evidence of primary tumor |
| Tis | Carcinoma in situ |
| | Squamous cell carcinoma in situ (SCIS) |
| | Adenocarcinoma in situ (AIS): adenocarcinoma with pure lepidic pattern, ≤3 cm in |
| | greatest dimension |
| T1 | Tumor ≤3 cm in greatest dimension, surrounded by lung or visceral pleura, without |
| | bronchoscopic evidence of invasion more proximal than the lobar bronchus (i.e., not in the |
| | main bronchus) |
| T1mi | Minimally invasive adenocarcinoma: adenocarcinoma (≤3 cm in greatest dimension) with |
| | a predominantly lepidic pattern and ≤5 mm invasion in greatest dimension |
| T1a | Tumor ≤1 cm in greatest dimension. A superficial, spreading tumor of any size whose |
| | invasive component is limited to the bronchial wall and may extend proximal to the main |
| | bronchus also is classified as T1a, but those tumors are uncommon. |
| T1b | Tumor >1 cm but ≤2 cm in greatest dimension |
| T1c | Tumor >2 cm but ≤3 cm in greatest dimension |
| T2 | Tumor >3 cm but ≤5 cm or having any of the following features: |
| | Involves the main bronchus regardless of distance to the carina, but without |
| | involvement of the carina |
| | Invades visceral pleura (PL1 or PL2) |
| | Associated with atelectasis or obstructive pneumonitis extending to the hilar |
| | region, involving part or all of the lung |
| | T2 tumors with these features are classified as T2a if ≤4 cm or if the size cannot be |
| | determined and T2b if >4 cm but ≤5 cm |
| T2a | Tumor >3 cm but ≤4 cm in greatest dimension |
| T2b | Tumor >4 cm but ≤5 cm in greatest dimension |
| T3 | Tumor >5 cm but ≤7 cm in greatest dimension or directly invading any of the following: |
| | parietal pleural (PL3), chest wall (including superior sulcus tumors), phrenic nerve, |
| | parietal pericardium; or separate tumor nodule(s) in the same lobe as the primary |
| T4 | Tumor >7 cm or tumor of any size that invades one or more of the following: diaphragm, |
| | mediastinum, heart, great vessels, trachea, recurrent laryngeal nerve, esophagus, vertebral |
| | body, or carina; or separate tumor nodule(s) in an ipsilateral lobe different from that of the |
| | primary |

Non-Small Cell Lung Cancer Regional lymph nodes (N)

| | |
|---|---|
| NX | Regional lymph nodes cannot be assessed |
| N0 | No regional node metastasis |
| N1 | Metastasis in ipsilateral peribronchial and/or ipsilateral hilar lymph nodes and |
| | intrapulmonary nodes, including involvement by direct extension |
| N2 | Metastasis in ipsilateral mediastinal and/or subcarinal lymph node(s) |
| N3c | Metastasis in the contralateral mediastinal, contralateral hilar, ipsilateral or contralateral |
| | scalene, or supraclavicular lymph node(s) |

Non-Small Cell Lung Cancer Distant metastasis (M)

| | |
|---|---|
| M0 | No distant metastasis |
| M1 | Distant metastasis |
| M1a | Separate tumor nodule(s) in a contralateral lobe tumor; tumor with pleural or pericardial |
| | nodules or malignant pleural or pericardial effusion. Most pleural (pericardial) effusion |
| | with lung cancer are a result of the tumor. In a few patients, however, multiple microscopic |
| | examinations of pleural (pericardial) fluid are negative for tumor, and the fluid is |
| | nonbloody and not an exudate. If these elements and clinical judgment dictate that the |
| | effusion is not related to the tumor, the effusion should be excluded as a staging descriptor. |
| M1b | Single extrathoracic metastasis in a single organ and involvement of a single nonregional |
| | node |
| M1c | Multiple extrathoracic metastases in a single organ or in multiple organs |

TABLE 22
Non-Small Cell Lung Cancer Anatomic stage/prognostic groups

| Stage | T | N | M |
|---|---|---|---|
| 0 | Tis | N0 | M0 |
| | T1mi | N0 | M0 |
| IA1 | T1a | N0 | M0 |
| IA2 | T1b | N0 | M0 |
| IA3 | T1c | N0 | M0 |
| IB | T2a | N0 | M0 |
| IIA | T2b | N0 | M0 |
| IIB | T1a | N1 | M0 |
| | T1b | N1 | M0 |
| | T1c | N1 | M0 |
| | T2a | N1 | M0 |
| | T2b | N1 | M0 |
| | T3 | N0 | M0 |
| | T1a | N2 | M0 |
| | T1b | N2 | M0 |
| | T1c | N2 | M0 |
| | T2a | N2 | M0 |
| IIIA | T2b | N2 | M0 |
| | T3 | N1 | M0 |
| | T4 | N0 | M0 |
| | T4 | N1 | M0 |
| IIIB | T1a | N3 | M0 |
| | T1b | N3 | M0 |
| | T1c | N3 | M0 |
| | T2a | N3 | M0 |
| | T2b | N3 | M0 |
| | T3 | N2 | M0 |
| | T4 | N2 | M0 |
| | T3 | N3 | M0 |
| IIIC | T4 | N3 | M0 |
| IVA | T Any | N Any | M1a |
| T Any | | N Any | M1b |
| IVB | T Any | N Any | M1c |

TABLE 23
Small Cell Lung Cancer Primary tumor (T)

| | |
|---|---|
| TX | Primary tumor cannot be assessed, or tumor is proven by the presence of malignant cells in |
| | sputum or bronchial washings but not visualized by imaging or bronchoscopy |
| TC | No evidence of primary tumor |
| Tis | Carcinoma in situ |
| | Squamous cell carcinoma in situ (SCIS) |
| | Adenocarcinoma in situ (AIS): adenocarcinoma with pure lepidic pattern, ≤3 cm in |
| | greatest dimension |
| T1 | Tumor ≤3 cm in greatest dimension, surrounded by lung or visceral pleura, without |
| | bronchoscopic evidence of invasion more proximal than the lobar bronchus (i.e., not in the |
| | main bronchus) |
| T1mi | Minimally invasive adenocarcinoma: adenocarcinoma (≤3 cm in greatest dimension) with |
| | a predominantly lepidic pattern and ≤5 mm invasion in greatest dimension |
| T1a | Tumor ≤1 cm in greatest dimension. A superficial, spreading tumor of any size whose |
| | invasive component is limited to the bronchial wall and may extend proximal to the main |
| | bronchus also is classified as T1a, but those tumors are uncommon. |
| T1b | Tumor >1 cm but ≤2 cm in greatest dimension |
| T1c | Tumor >2 cm but ≤3 cm in greatest dimension |
| T2 | Tumor >3 cm but ≤5 cm or having any of the following features: |
| | Involves the main bronchus regardless of distance to the carina, but without |
| | involvement of the carina |
| | Invades visceral pleura (PL1 or PL2) |
| | Associated with atelectasis or obstructive pneumonitis extending to the hilar |
| | region, involving part or all of the lung |
| | T2 tumors with these features are classified as T2a if ≤4 cm or if the size cannot be |
| | determined and T2b if >4 cm but ≤5 cm |
| T2a | Tumor >3 cm but ≤4 cm in greatest dimension |
| T2b | Tumor >4 cm but ≤5 cm in greatest dimension |
| T3 | Tumor >5 cm but ≤7 cm in greatest dimension or directly invading any of the following: |
| | parietal pleural (PL3), chest wall (including superior sulcus tumors), phrenic nerve, |
| | parietal pericardium; or separate tumor nodule(s) in the same lobe as the primary |
| T4 | Tumor >7 cm or tumor of any size that invades one or more of the following: diaphragm, |
| | mediastinum, heart, great vessels, trachea, recurrent laryngeal nerve, esophagus, vertebral |
| | body, or carina; or separate tumor nodule(s) in an ipsilateral lobe different from that of the |
| | primary |

Small Cell Lung Cancer Regional lymph nodes (N)

| | |
|---|---|
| NX | Regional lymph nodes cannot be assessed |
| N0 | No regional lymph node metastasis |
| N1 | Metastasis to ipsilateral peribronchial and/or ipsilateral hilar lymph nodes and |
| | intrapulmonary nodes, including involvement by direct extension |
| N2 | Metastases in ipsilateral mediastinal and/or subcarinal lymph node(s) |
| N3 | Metastasis in contralateral mediastinal, contralateral hilar, ipsilateral or contralateral |
| | scalene, or supraclavicular lymph node(s) |

Small Cell Lung Cancer Distant metastasis (M)

| | |
|---|---|
| M0 | No distant metastasis |
| M1 | Distant metastases |
| M1a | Separate tumor nodule(s) in a contralateral lobe tumor; tumor with pleural or pericardial |
| | nodules or malignant pleural or pericardial effusion. Most pleural (pericardial) effusion |
| | with lung cancer are a result of the tumor. In a few patients, however, multiple microscopic |
| | examinations of pleural (pericardial) fluid are negative for tumor, and the fluid is |
| | nonbloody and not an exudate. If these elements and clinical judgment dictate that the |
| | effusion is not related to the tumor, the effusion should be excluded as a staging descriptor. |
| M1b | Single extrathoracic metastasis in a single organ and involvement of a single nonregional |
| | node |
| M1c | Multiple extrathoracic metastases in a single organ or in multiple organs |

TABLE 24
Small Cell Lung Cancer Anatomic stage/prognostic groups

| | | | |
|---|---|---|---|
| Stage | T | N | M |

Limited disease

| | | | |
|---|---|---|---|
| 0 | Tis | N0 | M0 |
| | T1mi | N0 | M0 |
| IA1 | T1a | N0 | M0 |
| IA2 | T1b | N0 | M0 |
| IA3 | T1c | N0 | M0 |
| IB | T2a | N0 | M0 |
| IIA | T2b | N0 | M0 |
| IIB | T1a | N1 | M0 |
| | T1b | N1 | M0 |
| | T1c | N1 | M0 |
| | T2a | N1 | M0 |
| | T2b | N1 | M0 |
| | T3 | N0 | M0 |
| | T1a | N2 | M0 |
| | T1b | N2 | M0 |
| | T1c | N2 | M0 |
| IIIA | T2a | N2 | M0 |
| | T2b | N2 | M0 |
| | T3 | N1 | M0 |
| | T4 | N0 | M0 |
| | T4 | N1 | M0 |
| IIIB | T1a | N3 | M0 |
| | T1b | N3 | M0 |
| | T1c | N3 | M0 |
| | T2a | N3 | M0 |
| | T2b | N3 | M0 |
| | T3 | N2 | M0 |
| | T4 | N2 | M0 |
| IIIC | T3 | N3 | M0 |

Extensive disease

| | | | |
|---|---|---|---|
| IVA | T Any | N Any | M1a |
| | T Any | N Any | M1b |
| IVB | T Any | N Any | M1c |

TABLE 25
Breast Cancer Primary tumor (T)

| | |
|---|---|
| TX | Primary tumor cannot be assessed |
| T0 | No evidence of primary tumor |
| Tis | Carcinoma in situ |
| Tis (DCIS) | Ductal carcinoma in situ |
| Tis | Paget disease of the nipple NOT associated with invasive carcinoma and/or carcinoma in |
| (Paget) | situ (DCIS) in the underlying breast parenchyma. Carcinomas in the breast parenchyma |
| | associated with Paget disease are categorized on the basis of the size and characteristics of |
| | the parenchymal disease, although the presence of Paget disease should still be noted |
| T1 | Tumor ≤20 mm in greatest dimension |
| T1mi | Tumor ≤1 mm in greatest dimension |
| T1a | Tumor >1 mm but ≤5 mm in greatest dimension (round any measurement >1.0-1.9 mm to |
| | 2 mm) |
| T1b | Tumor >5 mm but ≤10 mm in greatest dimension |
| T1c | Tumor >10 mm but ≤20 mm in greatest dimension |
| T2 | Tumor >20 mm but ≤50 mm in greatest dimension |
| T3 | Tumor >50 mm in greatest dimension |
| T4 | Tumor of any size with direct extension to the chest wall and/or to the skin (ulceration or |
| | skin nodules), not including invasion of dermis alone |
| T4a | Extension to chest wall, not including only pectoralis muscle adherence/invasion |
| T4b | Ulceration and/or ipsilateral satellite nodules and/or edema (including peaud'orange) of |
| | the skin, which do not meet the criteria for inflammatory carcinoma |
| T4c | Both T4a and T4b |
| T4d | Inflammatory carcinoma |

Breast Cancer Regional lymph nodes (N)
Clinical

| | |
|---|---|
| cNX | Regional lymph nodes cannot be assessed (e.g., previously removed) |
| cN0 | No regional lymph node metastasis (on imaging or clinical examination) |
| cN1 | Metastasis to movable ipsilateral level I, II axillary lymph node(s) |
| cN1mi | Micrometastases (approximately 200 cells, larger than 0.2 mm, but none larger than 2.0 |
| | mm) |
| cN2 | Metastases in ipsilateral level I, II axillary lymph nodes that are clinically fixed or matted; |
| | or in ipsilateral internal mammary nodes in the absence of clinically evident axillary |
| | lymph node metastases |
| cN2a | Metastases in ipsilateral level I, II axillary lymph nodes fixed to one another (matted) or to |
| | other structures |
| cN2b | Metastases only in ipsilateral internal mammary nodes and in the absence of axillary |
| | lymph node metastases |
| cN3 | Metastases in ipsilateral infraclavicular (level III axillary) lymph node(s), with or without |
| | level I, II axillary node involvement, or in ipsilateral internal mammary lymph node(s) |
| | with level I, II axillary lymph node metastasis; or metastases in ipsilateral supraclavicular |
| | lymph node(s), with or without axillary or internal mammary lymph node involvement |
| cN3a | Metastasis in ipsilateral infraclavicular lymph node(s) |
| cN3b | Metastasis in ipsilateral internal mammary lymph node(s) and axillary lymph node(s) |
| cN3c | Metastasis in ipsilateral supraclavicular lymph node(s) |

Breast Cancer Pathologic (pN)

| | |
|---|---|
| pNX | Regional lymph nodes cannot be assessed (for example, previously removed, or not |
| | removed for pathologic study) |
| pN0 | No regional lymph node metastasis identified histologically, or isolated tumor cell clusters |
| | (ITCs) only. Note: ITCs are defined as small clusters of cells ≤ 0.2 mm, or single tumor |
| | cells, or a cluster of < 200 cells in a single histologic cross-section; ITCs may be detected |
| | by routine histology or by immunohistochemical (IHC) methods; nodes containing only |
| | ITCs are excluded from the total positive node count for purposes of N classification but |
| | should be included in the total number of nodes evaluated |
| pN0(i) | No regional lymph node metastases histologically, negative IHC |
| pN0(i+) | ITCs only in regional lymph node(s) |
| pN0(mol−) | No regional lymph node metastases histologically, negative molecular findings (reverse |
| | transcriptase polymerase chain reaction [RT-PCR]) |
| pN0(mol+) | Positive molecular findings by RT-PCR; no ITCs detected |
| pN1 | Micrometastases; or metastases in 1-3 axillary lymph nodes and/or in internal mammary |
| | nodes; and/or in clinically negative internal mammary nodes with micrometastases or |
| | macrometastases by sentinel lymph node biopsy |
| pN1mi | Micrometastases (200 cells, >0.2 mm but none >2.0 mm) |
| pN1a | Metastases in 1-3 axillary lymph nodes (at least 1 metastasis >2.0 mm) |
| pN1b | Metastases in ipsilateral internal mammary lymph nodes, excluding ITCs, detected by |
| | sentinel lymph node biopsy |
| pN1c | Metastases in 1-3 axillary lymph nodes and in internal mammary sentinel nodes (i.e., pN1a |
| | and pN1b combined) |
| pN2 | Metastases in 4-9 axillary lymph nodes; or positive ipsilateral internal mammary lymph |
| | nodes by imaging in the absence of axillary lymph node metastases |
| pN2a | Metastases in 4-9 axillary lymph nodes (at least 1 tumor deposit >2.0 mm) |
| pN2b | Clinically detected*¹ metastases in internal mammary lymph nodes with or without |
| | microscopic confirmation; with pathologically negative axillary lymph nodes |
| pN3 | Metastases in ≥10 axillary lymph nodes; or in infraclavicular (level III axillary) lymph |
| | nodes; or positive ipsilateral internal mammary lymph nodes by imaging in the presence of |
| | one or more positive level I, II axillary lymph nodes; or in >3 axillary lymph nodes and |
| | micrometastases or macrometastases by sentinel lymph node biopsy in clinically negative |
| | ipsilateral internal mammary lymph nodes; or in ipsilateral supraclavicular lymph nodes |
| pN3a | Metastases in ≥10 axillary lymph nodes (at least 1 tumor deposit >2.0 mm); or metastases |
| | to the infraclavicular (level III axillary lymph) nodes |
| pN3b | pN1a or pN2a in the presence of cN2b (positive internal mammary nodes by imaging) or |
| | pN2a in the presence of pN1b |
| pN3c | Metastases in ipsilateral supraclavicular lymph nodes |

Breast Cancer Distant metastasis (M)

| | |
|---|---|
| M0 | No clinical or radiographic evidence of distant metastasis |
| cM0(i+) | No clinical or radiographic evidence of distant metastases in the presence of tumor cells or |
| | deposits no larger than 0.2 mm detected microscopically or by molecular techniques in |
| | circulating blood, bone marrow, or other nonregional nodal tissue in a patient without |
| | symptoms or signs of metastasis |
| cM1 | Distant metastases detected by clinical and radiographic approaches |
| pM1 | Any histologically proven metastases in distant organs; or if in non-regional nodes, |
| | metastases >0.2 mm |

TABLE 26
Breast Cancer Histologic grade (G)

| | |
|---|---|
| GX | Grade cannot be assessed |
| G1 | Low combined histologic grade (favorable) |
| G2 | Intermediate combined histologic grade (moderately favorable) |
| G3 | High combined histologic grade (unfavorable) |

TABLE 27
Breast Cancer Anatomic stage/prognostic groups

| Stage | T | N | M |
|---|---|---|---|
| 0 | Tis | N0 | M0 |
| IA | T1 | N0 | M0 |
| IB | T0 | N1mi | M0 |
| | T1 | N1mi | M0 |
| IIA | T0 | N1 | M0 |
| | T1 | N1 | M0 |
| | T2 | N0 | M0 |
| IIB | T2 | N1 | M0 |
| | T3 | N0 | M0 |
| IIIA | T0 | N2 | M0 |
| | T1 | N2 | M0 |
| | T2 | N2 | M0 |
| | T3 | N1 | M0 |
| | T3 | N2 | M0 |
| IIIB | T4 | N0 | M0 |
| | T4 | N1 | M0 |
| | T4 | N2 | M0 |
| IIIC | Any T | N3 | M0 |
| IV | Any T | Any N | M1 |

Methods provided herein, in certain aspects, allow for early detection cancer or for detection of non-metastatic cancer. Examples of cancers that may be detected in accordance with a method disclosed herein include, without limitation, Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia,Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

Computer systems

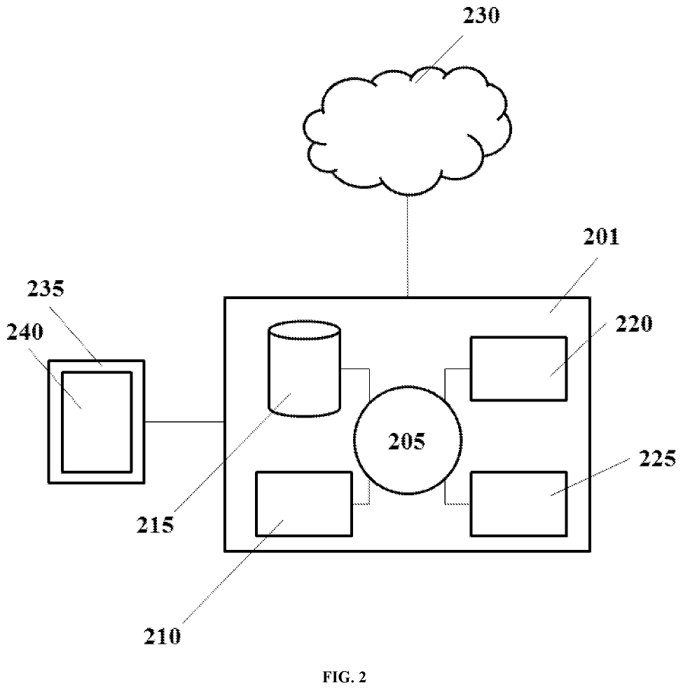

The present disclosure provides computer systems that are programmed to implement methods of the disclosure. FIG. 2 shows a computer system 201 that is programmed or otherwise configured to implement methods of the present disclosure. The computer system 201 can regulate various aspects of methods of the present disclosure, such as, for example, methods for determining that a subject has or is at risk of having a disease (e.g., cancer).

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., a healthcare provider or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, results of methods of the present disclosure. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can be, for example, a trained algorithm (or trained machine learning algorithm), such as, for example, a support vector machine or neural network.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Obtaining cfDNA Size Distribution

Circularization of single strand cfDNA:

12 μl of purified cfDNA fragments is denatured by heating at 95° C. for 30 seconds and chilling on ice for 2 minutes. Then, 8μl of ligation mix containing 2 μl of 10× CircLigase buffer, 4 μl of 5M Betaine, 1 μl of 50 mM MnCl2, and 1 μl of CircLigase II is added to the denatured DNA samples and the reactions are incubated at 60° C. for one hour.

Rolling cycle amplification of circular target polynucleotides:

For each reaction, add 5 μl of isothermal amplification buffer, 0.75uL of dNTP mix (25mM each), 2μl of 10 uM gene specific primers, 20.25 μl of water. Heat the reaction at 80° C. for 1 minute and incubated at 63oC for 5 minutes before cooling down to 4° C. Next, add 15 units of Bst 2.0 warm start DNA polymerase to each reaction, and incubated in a thermal cycler with the following program: 4 cycles of 60° C. for 30 seconds; 70° C. for 4.5 minutes; 94° C. for 20 seconds; and 58° C. for 10 seconds.

2nd round of PCR and sequencing:

Purify rolling cycle amplification products by addition of 45 μl Ampure beads, following the manufacturer's instructions for the remaining wash steps. Elute in 25 μl of elution buffer. Further amplify the purified RCA products by PCR with primers containing sequencing adapters. Sequence the resulting amplification products by NGS.

NGS data analysis:

Align FASTQ files to a reference file containing the target sequence.

### Example 2

### Comparison of cfDNA Sequence Reads and Breakpoint Sequences from Healthy and CRC Samples of Different Cancer Stages

cfDNA from 426 of healthy individuals, and 197 CRC patients (8 stage, 32 stage I, 64 stage II, 69 stage III, 24 stage IV) are circularized and amplified as described in a protocol, using a panel of primers targeting genes.

Sequence reads and breakpoint sequences are compared to reference sequence reads and reference breakpoint sequences. Sequence reads and breakpoint sequences corresponding to reference sequence reads and reference breakpoint sequences are identified. Individuals with cancer have a greater percentage of sequence reads and breakpoint sequences corresponding to reference sequence reads and reference breakpoint sequences than the healthy controls (at least 0.1% in individuals with cancer compared to less than 0.01% in individuals without cancer).

### Example 3

### Screening Non-Metastatic Cancer

An individual presents at a clinic for a routine checkup. A blood sample is taken from the individual to determine whether they have or are at risk for developing early stage cancer. Cell-free DNA (cfDNA) is isolated from the sample and then amplified prior to sequencing. The individual shows an increase of cfDNA sequence reads corresponding to reference cfDNA sequence reads indicating that the individual has early stage or non-metastatic cancer or that they are at risk of developing cancer. The individual is advised to seek care with an oncologist who can assess the state or stage of cancer. The individual is treated for the cancer and survives cancer free for 10 years.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for determining that a subject has or is at risk of having a disease, comprising:
(a) obtaining a cell-free biological sample of said subject;
(b) using a plurality of nucleic acid molecules derived from said cell-free biological sample to generate a plurality of circular nucleic acid molecules;
(c) subjecting said plurality of circular nucleic acid molecules or derivatives thereof to sequencing to generate a plurality of sequence reads; and
(d) processing said plurality of sequence reads against a plurality of reference sequence reads to identify at least a subset of said plurality sequence reads as corresponding to at least a subset of said plurality of reference sequence reads, thereby determining that said subject has or is at risk of having said disease.

2. A method for determining that a subject has or is at risk of having a disease, comprising:
(a) obtaining a cell-free biological sample of said subject;
(b) using a plurality of nucleic acid molecules derived from said cell-free biological sample to generate a plurality of circular nucleic acid molecules;
(c) subjecting said plurality of circular nucleic acid molecules or derivatives thereof to sequencing to identify a plurality of breakpoint sequences; and
(d) processing said plurality of breakpoint sequences against a plurality of reference breakpoint sequences to identify at least a subset of said plurality breakpoint sequences as corresponding to at least a subset of said plurality of reference breakpoint sequences, thereby determining that said subject has or is at risk of having said disease.

3. The method of claim 2, wherein each of said plurality of breakpoint sequences comprises a 5′ end and a 3′ end of a nucleic acid molecule derived from said cell-free biological sample.

4. The method of claim 1 or claim 2, wherein (b) comprises ligating ends of each of said plurality of nucleic acid molecules to one another.

5. The method of claim 1 or claim 2, wherein (b) comprises coupling an adapter to a 3′ end, a 5′ end, or both a 5′end and a 3′end of each of said plurality of nucleic acid molecules.

6. The method of claim 1 or claim 2, further comprising, subsequent to (b), subjecting each of said plurality of circular nucleic acid molecules to nucleic acid amplification to generate a plurality of amplification products of said plurality of circular nucleic acid molecules, wherein (c) comprises subjecting said plurality of amplification products or derivatives thereof to sequencing to generate a plurality of sequence reads or a plurality of breakpoint sequences of said plurality of amplification products or derivatives thereof.

7. The method of claim 6, wherein said nucleic acid amplification is effected by a polymerase having strand-displacement activity.

8. The method of claim 6, wherein said nucleic acid amplification is effected by a polymerase that does not have strand-displacement activity.

9. The method of claim 6, wherein said nucleic acid amplification comprises contacting said circular nucleic acid molecule to an amplification reaction mixture comprising random primers.

10. The method of claim 6, wherein said nucleic acid amplification comprises contacting said circular nucleic acid molecule to an amplification reaction mixture comprising one or more primers, each of which specifically hybridizes to a different target sequence via sequence complementarity.

11. The method of claim 1 or claim 2, wherein said plurality of nucleic acid molecules is single stranded.

12. The method of claim 1 or claim 2, wherein said plurality of nucleic acid molecules comprises cell-free DNA or RNA.

13. The method of claim 1 or claim 2, wherein said plurality of nucleic acid molecules is from a tumor.

14. The method of claim 1 or claim 2, further comprising processing said sequence to monitor a progression or regression of said disease in said subject in response to treatment.

15. The method of claim 1 or claim 2, wherein said sequencing comprises a method selected from one or more of sequencing by synthesis, sequencing by ligation, nanopore sequencing, nanoball sequencing, ion detection, sequencing by hybridization, polymerized colony (POLONY) sequencing, nanogrid rolling circle sequencing (ROLONY), and ion torrent sequencing.

16. The method of claim 1 or claim 2, wherein said cell-free biological sample comprises less than 75 nanograms of nucleic acids.

17. The method of claim 1 or claim 2, wherein said cell-free biological sample comprises a bodily fluid.

18. The method of claim 17, wherein said bodily fluid is urine, saliva, blood, serum, plasma, tears, sputum, cerebrospinal fluid, synovial fluid, mucus, bile, semen, lymph, amniotic fluid, menstrual fluid, or combinations thereof.

19. The method of claim 1 or claim 2, further comprising computer processing said sequence to identify an epigenetic modification in said sequence.

20. The method of claim 19, wherein said epigenetic modification is selected from methylation, phosphorylation, ubiquitination, sumoylation, acetylation, ribosylation, citrullination, and fragmentation.

21. The method of claim 1 or 2, wherein said disease is cancer.

22. The method of claim 21, wherein said cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, gastric cancer, ovarian cancer, pancreatic cancer, brain cancer, leukemia, lymphoma, and myeloma.

23. The method of claim 1, further comprising using at least said subset of said plurality sequence reads identified in (d) to output an electronic report indicating that said subject has or is at risk of having said disease.

24. The method of claim 2, further comprising using at least said subset of said plurality breakpoint sequences identified in (d) to output an electronic report indicating that said subject has or is at risk of having said disease.

25. The method of claim 1, further comprising using at least said subset of said plurality sequence reads identified in (d) to provide a therapeutic intervention to said subject for said disease.

26. The method of claim 2, further comprising using at least said subset of said plurality breakpoint sequences identified in (d) to provide a therapeutic intervention to said subject for said disease.

27. The method of claim 1, further comprising using at least said subset of said plurality sequence reads identified in (d) to treat said subject for said disease.

28. The method of claim 2, further comprising using at least said subset of said plurality breakpoint sequences identified in (d) to treat said subject for said disease.

29. The method of claim 27 or 28, wherein said subject is treated by administering a chemotherapy or immunotherapy to said subject.

30. The method of any one of claims 23 to 28, wherein said disease is cancer.

31. The method of claim 1, further comprising using at least said subset of said plurality sequence reads identified in (d) to monitor said subject for a progression or regression of said subject.

32. The method of claim 2, further comprising using at least said subset of said plurality breakpoint sequences identified in (d) to monitor said subject for a progression or regression of said subject.

33. The method of claim 1, wherein (d) comprises determining that at least 0.1% of said plurality of sequence reads correspond to said plurality of reference sequence reads.

34. The method of claim 2, wherein (d) comprises determining that at least 0.1% of said plurality of breakpoint sequences correspond to said plurality of reference breakpoint sequences.

35. A method for determining that a subject has or is at risk of having a disease, comprising:
(a) accessing (i) a first database comprising a plurality of sequence reads, wherein a sequence read of said plurality of sequence reads comprises multiple copies of a sequence of a nucleic acid molecule from said subject; and (ii) a second database comprising a plurality of reference sequence reads; and
(b) processing said plurality of sequence reads from said first database against said plurality of reference sequence reads from said second database to identify at least a subset of said plurality sequence reads as corresponding to at least a subset of said plurality of reference sequence reads, thereby determining that said subject has or is at risk of having said disease.

36. A method for determining that a subject has or is at risk of having a disease, comprising:
(a) accessing (i) a first database comprising a plurality of breakpoint sequences, wherein a breakpoint sequence of said plurality of breakpoint sequences comprises multiple copies of a sequence of a 5′ end and a 3′ end of a nucleic acid molecule of said subject; and (ii) a second database comprising a plurality of reference breakpoint sequences; and
(b) processing said plurality of breakpoint sequences from said first database against said plurality of reference breakpoint sequences from said second database to identify at least a subset of said plurality breakpoint sequences as corresponding to at least a subset of said plurality of reference breakpoint sequences, thereby determining that said subject has or is at risk of having said disease.

37. The method of claim 35 or claim 36, wherein said first database and said second database are different databases.

38. The method of claim 35 or claim 36, wherein said disease is cancer.

39. A system for determining that a subject has or is at risk of having a disease, comprising:
(a) a first database comprising a plurality of sequence reads, wherein a sequence read of said plurality of sequence reads comprises multiple copies of a sequence of a nucleic acid molecule of said subject;
(b) a second database comprising a plurality of reference sequence reads; and
(c) one or more computer processors that are individually or collectively programed to process said plurality of sequence reads from said first database against said plurality of reference sequence reads from said second database to identify at least a subset of said plurality sequence reads as corresponding to at least a subset of said plurality of reference sequence reads, thereby determining that said subject has or is at risk of having said disease.

40. A system for determining that a subject has or is at risk of having a disease, comprising:
(a) a first database comprising a plurality of breakpoint sequences, wherein a breakpoint sequence of said plurality of breakpoint sequences comprises multiple copies of a sequence of a 5′ end and a 3′ end of a nucleic acid molecule of said subject;
(b) a second database comprising a plurality of reference breakpoint sequences; and
(c) one or more computer processors that are individually or collectively programed to process said plurality of breakpoint sequences from said first database against said plurality of reference breakpoint sequences from said second database to identify at least a subset of said plurality breakpoint sequences as corresponding to at least a subset of said plurality of reference breakpoint sequences, thereby determining that said subject has or is at risk of having said disease.

41. The system of claim 39 or claim 40, wherein said first database and said second database are different databases.

42. The system of claim 39 or claim 40, wherein said disease is cancer.

43. A non-transitory computer-readable medium comprising machine-executable code that, upon execution by one or more computer processors, implements a method for determining that a subject has or is at risk of having a disease, comprising:
(a) accessing (i) a first database comprising a plurality of sequence reads, wherein a sequence read of said plurality of sequence reads comprises multiple copies of a sequence of a nucleic acid molecule from said subject; and (ii) a second database comprising a plurality of reference sequence reads; and
(b) processing said plurality of sequence reads from said first database against said plurality of reference sequence reads from said second database to identify at least a subset of said plurality sequence reads as corresponding to at least a subset of said plurality of reference sequence reads, thereby determining that said subject has or is at risk of having said disease.

44. A non-transitory computer-readable medium comprising machine-executable code that, upon execution by one or more computer processors, implements a method for determining that a subject has or is at risk of having a disease, comprising:
(a) accessing (i) a first database comprising a plurality of breakpoint sequences, wherein a breakpoint sequence of said plurality of breakpoint sequences comprises multiple copies of a sequence of a 5′ end and a 3′ end of a nucleic acid molecule of said subject; and (ii) a second database comprising a plurality of reference breakpoint sequences; and
(b) processing said plurality of breakpoint sequences from said first database against said plurality of reference breakpoint sequences from said second database to identify at least a subset of said plurality breakpoint sequences as corresponding to at least a subset of said plurality of reference breakpoint sequences, thereby determining that said subject has or is at risk of having said disease.

45. The non-transitory computer-readable medium of claim 43 or claim 44, wherein said first database and said second database are different databases.

46. The non-transitory computer-readable medium of claim 43 or claim 44, wherein said disease is cancer.
